(19) United States
(12) Patent Application Publication
(10) Pub. No.: US 2011/0091931 A1
(43) Pub. Date: Apr. 21, 2011

(54) Methods and Kits for Determining Oxygen Free Radical (OFR) Levels in Animal and Human Tissues as a Prognostic Marker for Cancer and Other Pathophysiologies

(75) Inventors: Hirak S. Basu, Madison, WI (US); David Zarling, Menlo Park, CA (US)

(73) Assignee: COLBY PHARMACEUTICAL COMPANY, Menlo Park, CA (US)

(21) Appl. No.: 12/766,781

(22) Filed: Apr. 23, 2010

Related U.S. Application Data
(60) Provisional application No. 61/172,308, filed on Apr. 24, 2009.

(51) Int. Cl. C12Q 1/04

(52) U.S. Cl. 435/34

(57) ABSTRACT

Described herein are methods and techniques for the determination of reactive oxygen species in resected animal and human tissues. Also described herein are methods for determining the efficacy of anti-oxidant, oxidative stress modulatory, or anti-inflammatory drug chemoprevention or chemotherapy for the prevention and/or therapy of chronically inflamed and/or progressive cancers and pathophysiologies.

### CROSS-REFERENCE

This application claims the benefit of U.S. provisional application Ser. No. 61/172,308 filed Apr. 24, 2009, which are incorporated by reference in its entirety.

### FIELD OF THE INVENTION

Described herein are methods and techniques for the determination of reactive oxygen species in animal and human tissues.

### BACKGROUND OF THE INVENTION

Diagnosis followed by proper surgical and/or radiotherapy intervention is often the method of treatment for most solid tumors, including prostate cancer (CaP). As many CaP patients have an indolent or inflammatory or virulent form of the disease that is either non-fatal or chronic non-resolving inflammatory or virulent and fatal, a prognostic indicator of CaP that can differentiate between the indolent and the chronic non-resolving inflammatory and virulent forms of the disease, along with an effective CaP chemopreventive agent or vaccine or chemotherapeutic agent, including anti-inflammatory therapeutic intervention, should not only save patients from unnecessary treatment, but, should also help preserve the quality of life in a majority of the patients, who may need minimal therapeutic intervention.

Reactive oxygen species (ROS) are ions or very small molecules that include oxygen ions, free radicals, and peroxides, both inorganic and organic. They are highly reactive due to the presence of unpaired valence shell electrons. ROS form as a natural byproduct of the normal metabolism of oxygen and have important roles in cell signaling. However, during times of environmental stress, (such as, for example, UV- or heat-exposure), ROS levels can increase dramatically, which can result in significant damage to cell structures. This cumulates into a situation known as oxidative stress (OS). The ROS and ensuing OS are generated by endogenous metabolism and id also generated by exogenous sources, including ionizing radiation.

Reactive oxygen species (ROS) and the coupled oxidative stress (OS) have been associated with inflammation and tumor formation, tumor progression, tumor angiogenesis, tumor immune suppression, tumor cell proliferation and migration, tumor invasion of vasculative and other tissues, and tumor metastasis and resistance to all known therapeutics. Several studies suggested that ROS can act as secondary messengers and control various signaling cascades and inflammatory reactions. Research has indicated that oxidative stress is inherent in prostate cancer cells or can be induced by exogenous Dioxin (Agent Orange) and oxidative stress and inflammation and cell damage is required for an aggressive tumor phenotype and metastasis.

### SUMMARY OF THE INVENTION

Presented herein are methods for determining reactive oxygen species in animal and human tissues.

In one aspect is a method for quantitating the oxidative status in matched resected prostate tumor and normal prostate tissues from a patient pool of prostate cancer patients who underwent radical prostatectomy comprising;

providing at least one normal prostate tissue sample and at least one malignant tissue sample from the same patient; performing immuno-histochemistry (IHC) on the at least one normal prostate tissue sample and the at least one malignant prostate tissue sample; and quantitating the oxidative status using a fluorescence or other quantitation method.

In one embodiment, the patient pool of prostate cancer patients comprises between about 5 to about 500 patients. In another embodiment, the patient pool comprises between about 200 to about 250 patients. In a further embodiment, the patient pool comprises between about 225 to about 250 patients. In another embodiment, at least one normal prostate tissue sample and/or at least one malignant prostate tissue sample is in the form of an archival paraffin block. In a further embodiment, the at least one normal prostate tissue sample and/or at least one malignant prostate tissue sample is mounted on a glass slide or is contained in a tube or microplate format. In another embodiment, oxidative status is quantitated by measuring oxidative stress levels in the prostate gland or circulating prostate cancer cells. In a further embodiment, SSAT enzyme levels are an indicator of oxidative stress levels. In another embodiment, OH-⁸dGuanine levels in the DNA are an indicator of oxidative stress levels as a diagnosis of DNA oxidation. In yet another embodiment, an antibody is used to quantitate SSAT enzyme levels in the paraffin sample. In a further embodiment, the antibody is rabbit polyclonal IgG. In yet another embodiment, SSAT levels are detected using an IHC method. In yet another embodiment, OH-⁸dGuanine levels in the paraffin sample are detected using an IHC method with an antibody specifically directed to OH-⁸dGuanine. In a further embodiment, an antibody is used to detect OH-⁸dGuanine levels in the paraffin sample. In another embodiment, the antibody is highly specific mouse monoclonal or a specific polyclonal antibody. In yet another embodiment, the IHC of SSAT and OH-⁸dGuanine are performed in two adjacent sections of the same paraffin sample. In another embodiment, the fluorescence is detected and quantified using a fluorescence tagged secondary antibody using a fluorescence quantitation method. In a further embodiment, the fluorescence quantitation method is the AQUA or a similar method.

In another aspect is a method for prognosis comprising comparing cellular reactive oxygen species levels and the dynamics of sub-cellular reactive oxygen species distribution in resected patient tissues with a clinical outcome. In one embodiment is a method wherein the tissues are washed in PBS to remove blood cell contamination. In another embodiment, the tissues are sliced in sections. In a further embodiment, the sections are about 5 mm in thickness. In another embodiment, the tissues are soaked in a fluorescent indicator. In a further embodiment, the fluorescent indicator is HEt dye. In yet a further embodiment, the tissues are fixed and embedded in paraffin. In another embodiment, the tissues are analyzed for HEt fluorescence using a fluorescent quantitation method. In a further embodiment, the fluorescent method is the AQUA method. In a further embodiment the fluorescent method utilizes a BD Pathway Bioimager or an equivalent instrument using Hoechst 33342 dye DNA fluorescence as an internal control and the HE. In a further embodiment, the tissue samples are processed for IHC staining and AQUA analysis for SSAT and OH-⁸dGuanine estimation. In yet another embodiment, the tissues are dispersed into single cells and plated on multiple 96-well plates. In further embodiments the plates are stained with Hoechst 33342 or similar DNA binding dye. In yet further embodiments, the plates are stained for nuclear imaging in live cells. In other embodiments, a few of the plates are incubated with HEt dye for one hour at room or other temperatures. In another embodiment, the total reactive species level is determined using a BD Pathway Bioimager or an equivalent instrument. In yet a further embodiment, the kinetics of dye oxidation are determined by fluorescence intensity vs. time plot of subcellular organelles by segmentation analysis and data integration. In yet another embodiment, the dynamics of subcellular reactive oxygen species distribution is determined by the methods described herein with tissue plates kept at 37° C. air/CO2 incubator for about 4, about 12, about 24 and about 48 hours.

In another aspect is a method for determining changes in reactive oxygen species levels and sub-cellular reactive oxygen species distribution in resected prostate tumor and normal prostate tissues from patients enrolled in chemoprevention clinical trials of anti-oxidants, as neo-adjuvants, comprising collecting human tumor and normal prostate cells from prostate cancer patients undergoing neo-adjuvant clinical trials; and evaluating the neo-adjuvant levels, biologic response to the neo-adjuvant or markers of the neo-adjuvant activity. In one embodiment, the neo-adjuvant is an anti-oxidant. In another embodiment, the neo-adjuvant is 3,3′-diindolylmethane. In a further embodiment, the neo-adjuvant levels are 3,3′-diindolylmethane levels. In another embodiment the markers of neo-adjuvant activity are markers of 3,3′-diindolylmethane activity such as the androgen receptor, PSA, Ki-67, caspase-3, and 3,3′diindolylmethane-specific markers.

In one aspect is a method for differentiating prostate cancer tissues based upon oxidative stress status comprising:

obtaining a tissue sample;

fixing a tissue sample;

treating the tissue sample with a fluorescence indicator; and

analyzing the treated tissue sample with a fluorescence microscope.

In another embodiment is a method of standardizing the fluorescence quantitation of the cellular reactive oxygen species in a prostate tissue comprising:

fixing a tissue sample;

treating the tissue sample with a fluorescence indicator; and

analyzing the treated tissue sample with a fluorescence microscope.

In another embodiment is a method of determining the sub-cellular distribution of reactive oxygen species in a prostate tissue comprising:

fixing a tissue sample;

treating the tissue sample with a fluorescence indicator; and

analyzing the treated tissue sample with a fluorescence microscope.

In another embodiment is a method of determining the efficacy of anti-oxidant chemoprevention for the prevention of prostate cancer comprising:

fixing a tissue sample;

treating the tissue sample with a fluorescence indicator;

analyzing the treated tissue sample with a fluorescence microscope; and

comparing the sample fluorescence to a control sample.

In a further embodiment is the method wherein the fluorescence indicator is selected from dichlorofluorescein diacetate, or hydroethidine dye.

In a further embodiment is the method wherein the tissue sample is a non-resected or resected tissue sample.

In a further embodiment is the method wherein the analysis with a fluorescence microscope further comprises employment of the automated quantitative analysis system (AQUA).

In another embodiment is a method for differentiating prostate cancer tissues based upon oxidative stress status comprising:

treating a live animal with hydroethidine dye;

obtaining a tissue sample; and

analyzing the treated tissue sample with a fluorescence microscope.

In another embodiment is a method of standardizing the fluorescence quantitation of the cellular reactive oxygen species in a prostate cancer tissue comprising:

treating a human or live animal with hydroethidine dye;

obtaining a tissue sample; and

analyzing the treated tissue sample with a fluorescence microscope.

In another embodiment is a method of determining the sub-cellular distribution of reactive oxygen species in a prostate tissue comprising:

treating a live animal with hydroethidine dye;

obtaining a tissue sample; and

analyzing the treated tissue sample with a fluorescence microscope.

In another embodiment is a method of determining the efficacy of anti-oxidant chemoprevention for the prevention of prostate cancer comprising:

treating a live animal with hydroethidine dye;

obtaining a tissue sample; and

analyzing the treated tissue sample with a fluorescence microscope.

In another embodiment is a tissue sample comprising a tissue sample obtained from an animal or human, treated with a fluorescence indicator and fixed for analysis by a fluorescence microscope.

In another embodiment is the method wherein the fluorescence indicator is selected from dichlorofluorescein diacetate, or hydroethidine dye. In another embodiment is the method wherein the fluorescence indicator is hydroethidine dye.

In another embodiment is a tissue sample comprising a sample of tissue obtained after administration of a fluorescence indicator to a live animal, followed optionally by animal surgery or sacrifice, tissue extraction and processing for analysis by a fluorescence microscope. In one embodiment, the tissue extraction is via tissue biopsy.

In a further embodiment is a tissue sample comprising a paraffin block of resected prostate tissue treated with a fluorescent indicator. In another embodiment, the fluorescent indicator is responsive to reactive oxygen species. In a further embodiment, the resected prostate tissue is a malignant, hyperplastic, inflamed or normal human prostate tissue. In yet a further embodiment is an array of tissue samples comprising at least two tissue samples, wherein each tissue sample comprises a resected prostate tissue mounted on a paraffin block. In one embodiment, the each resected prostate tissue is treated with a fluorescent indicator. In a further embodiment, the array of tissue samples comprises a mixture of malignant and normal human prostate tissue. In a further embodiment, the array of tissue samples is from an animal.

Also described herein is a tissue sample database comprising a computer memory linked to a database comprising information generated from fluorescent analysis of prostate tissue samples. In one embodiment, the prostate tissue samples are malignant tumor and/or normal tissue samples. In a further embodiment, the prostate tissue samples have been treated with a fluorescent dye. In a further embodiment, the prostate tissue samples are analyzed using the AQUA method and/or an equivalent method thereof. In a further embodiment, the information generated from fluorescent analysis is related to the measurement of oxidative stress levels in the prostate tissue samples.

In a further embodiment is a kit for the quantitative ex vivo estimation of reactive oxygen species in a prostate tissue sample comprising a fluorescence indicator responsive to reactive oxygen species and instructions for use of the kit.

In a further embodiment is a kit for the quantitative ex vivo estimation of reactive oxygen species in a prostate tissue sample comprising a fluorescence indicator responsive to reactive oxygen species and instructions for analysis of the data.

In a further embodiment is a kit for the prediction of prostate inflammation and/or cancer virulence comprising a fluorescence indicator responsive to reactive oxygen species and instructions for use of the kit.

In a further embodiment is a kit for the prediction of prostate inflammation and/or cancer virulence comprising a fluorescence indicator responsive to reactive oxygen species and instructions for analysis of the data.

In another embodiment is the kit, wherein the fluorescence indicator is hydroethidine dye.

In another embodiment is the kit, wherein the instructions for analysis of the data further comprise a comparative dataset correlating concentration of reactive oxygen species to prostate cancer progression.

One embodiment provides the compounds of Formula Ia or Ib (structures given below).

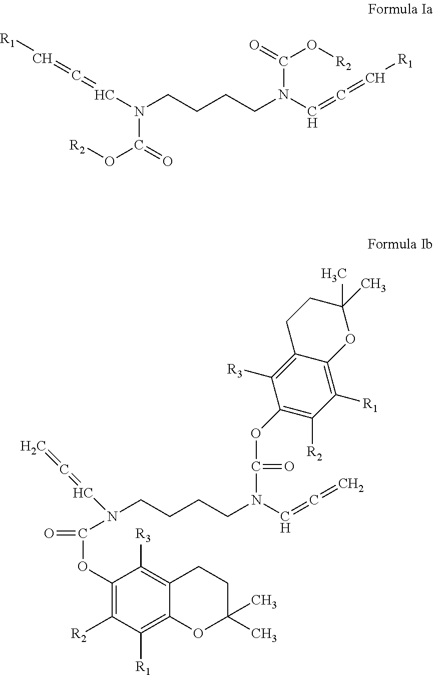

One embodiment provides a method to block androgen induced oxidative stress in androgen dependent LNCaP human prostate tumor cell comprising administration of a composition comprising a compound of Formula Ia or Ib.

Another embodiment provides a method of enhancing the treatment of prostate cancer comprising administration of a composition comprising a compound of Formula Ia or Ib.

The present disclosure provides variations of inflammatory and/or oxidized DNA repair, as well as other oxidation loci that are tightly linked to gene expression and demonstrates unusual patterns of population differentiation. Given the potential importance of genomic variations in the differential risk for diseases, and the invention provides an association of the variation within these locus with prostate cancer in population at risk, including the African-American, Asian-American, Native-American or European-American or other populations.

The findings presented herein can have an important impact on the design of clinical trials focused upon the prevention of prostate inflammation or cancers in subject populations, for example in high-risk individuals, on the implementation of programs aimed at early screening and timely treatment during the window of prevention or curability, or on individualized treatment of subjects with chronic inflammatory, progressive or advanced diseases. The above features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a Study Schema: Phase Ib placebo-controlled trial of diindolylmethane(DIM) in the study of the modulation of intermediate endpoint markers in patients with prostate cancer who are undergoing prostatectomy;

FIG. 2 illustrates DCF fluorescence (green) and Hoechst dye-DNA fluorescence (blue) in LNCaP cells at 20× magnification, (left)) Low oxidative stress in cells growing without androgen and (right) High oxidative stress in cells treated with 1 nM of the androgen analog metribolone for 96 h;

FIG. 3 illustrates nuclear and cytoplasmic contour maps of DCF dye fluorescence in LNCaP human prostate tumor cells;

FIG. 4 illustrates (a) plot of fluorescence intensity (F) vs time of DCFH dye oxidation in the nucleus of a representative LNCaP cell treated with 1 nM metribolone for 96 h, including the fitted curve (see text), and (b) residual of the fitted plot;

FIG. 5 shows a plot of initial velocities (v0, in Fluor. Unit/sec) for DCF dye oxidation in cytoplasm (A) and nucleus (B) of LNCaP cells exposed to a fixed concentration of metribolone for increasing time (the bars marked with *, + and ++ marks significant changes (p<0.05) compared to control or 48 h treatment respectively);

FIG. 6 shows IHC staining of the Androgen Receptor (AR) in LNCaP cells grown in 0.05 nM metribolone for 6 days (A) and I nM metribolone for 4 days (B) (fluorescence microscopy was carried out in an Olympus fluorescence microscope fitted with a Nikon digital camera: Magnification 100×);

FIG. 7 shows the plot of the mean and standard deviation of the ratio of nuclear fluorescence over cytoplasmic fluorescence in AR IHC slides of LNCaP cells treated with low (0.05 nM, control) or high (1 nM) concentrations of the androgen analog metribolone (the results are the mean of 45 and 39 cells from low and high metribolone treatment, respectively);

FIG. 8 shows (a) fluorescence micrograph of normal prostatic lumen of TRAMP animal treated with HEt (8 mg/kg) one hour before sacrifice, (b) the H&E picture of the same lumen (Magnification 40×) and (c) FIG. 8c. panels show H&E stained light, (A, top left & C, bottom left) and hydroethidine dye fluorescence (B, top right & D, bottom right) micrographs of mouse prostate tissue section from a 20 week old TRAMPxFVB mouse injected with 8 mg/kg Hydroethidine i.v. one hour before sacrifice; FIG. 8c Panels A, top left, & B, top right, show a prostate tissue section from the control, vehicle treated mouse and panels C, bottom left & D, bottom right, show prostate tissue of a mouse treated with CPC-200 drug (6 drug injections, once every 2 weeks); all these pictures in FIG. 8c were taken at 400× magnification using an Olympus BH-2 fluorescence microscope coupled with a Sony DSC V3 digital camera using 480 nm excitation/600 nm emission filters;

FIG. 9 shows (a) fluorescence micrograph of the normal prostatic lumen of a GTP treated TRAMP animal (see text) treated with HEt (8 mg/kg) one hour before sacrifice, and (b) Same lumen stained with H&E (Magnification 40);

FIG. 10 illustrates BD Pathway Bioimager or equivalent digital imaging microscopy of resected human prostate tissue (A) stained ex vivo with HEt dye and (B) stained ex vivo with Hoechst 33342 dye (see Methods for detail);

FIG. 11 illustrates the mean fluorescence intensity of HEt dye oxidation in normal and tumor cell nuclei (solid bar) and cytoplasms (grey bar), as determined by AQUA quantitation;

FIG. 12 shows one representative confocal section of the BD Bioimager of a human prostate tissue treated with HEt dye ex vivo and stained with Hoechst 33342, note the oxidized HEt is specifically staining the mitochondria as evident by the granular image of the cytoplasm;

FIG. 13 shows a table of total fluorescence of the epithelial tissue in control and the green tea polyphenol, ECGC, treated animals; and

FIG. 14 provides a table showing the total Het fluorescence of normal and cancerous epithelial cells and the corresponding Gleason score.

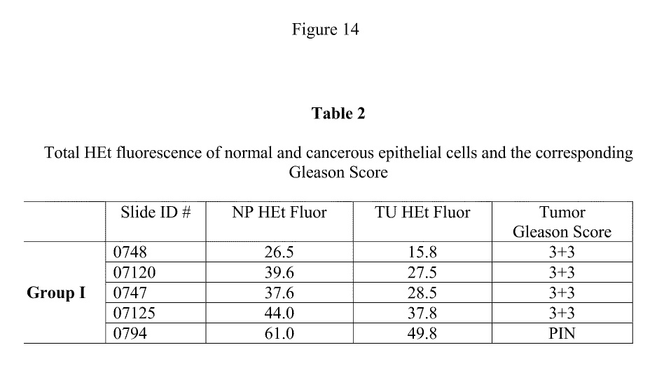

FIG. 15a shows the first Carbamate derivative of CPC-200 for oral administration. R₁ and R₂are alkyl groups with carbon chain length between 1-5 including isopropyl and isobutyl groups; and FIG. 15b shows the second Cabamate derivative of CPC-200 for oral administration where. R₁ and R₂ are alkyl groups with carbon chain length between 1-5 including isopropyl and isobutyl groups.

### DETAILED DESCRIPTION

100541 Advanced hormone refractory metastatic prostate cancer (CaP) is the second leading cause of cancer deaths among US men. In 2007, over 218,000 US men were diagnosed with CaP and over 27,000 men we re anticipated to die of this disease. Although about 17% of US men are likely to be diagnosed with CaP in their life-time, only 3% of them will ultimately die of this disease. Therefore, the development of a clinically useful method for differentiating indolent CaP from the chronic inflammatory and/or virulent disease at an early stage and establishing a non-toxic chemoprevention or chemotherapeutic strategy that delays or prevents the recurrence of the inflammatory or virulent CaP will have a major impact in the clinical management of this killer disease. Aberrant production and distribution of reactive oxygen species (ROS), such as H₂O₂, hydroxyl radicals, superoxide anions, etc. are found to be important in carcinogenesis as well as in regulating cancer progression, chronic inflammation and metastasis and in pathophysiological disease. Because of the relatively low systemic toxicity of antioxidant drugs as neo-adjuvants, it is often difficult to reach the dose limiting toxicity (DLT) and therefore, to determine appropriate dose and dosing regimen. Due to a lack of a clinically applicable method of determining tissue oxidative stress, there exists no good method of determining drug doses or predict drug effectiveness. In order to investigate changes in ROS in live cells, a BD Pathway 855 Bioimager or equivalent imaging system and data analysis software (Becton-Dickinson, San Jose, Calif.) are used to acquire, process and quantitate the fluorescence intensities of cellular and subcellular segments in live cells. The cells will be obtained from the resected human tissues or tissue biopsies. Using hydroethidine dye (HEt) that is oxidized by the ROS to highly fluorescent ethidium ion (E⁺), we have standardized a fluorescence microscopic method of observing ROS levels in prostate and other tissues in vivo. We have also standardized conditions for ex vivo HEt dye oxidation for estimating ROS levels in the prostate and other tissues resected from animals and humans. Here, in one embodiment is a method of standardizing the fluorescence quantitation of the cellular ROS and their subcellular distribution and translocation in the resected patient tissues or tissue biopsies. We have standardized methods for quantitation of ROS and their subcellular distributions. These methods are used in order to establish a reliable pharmacodynamics of drugs in chemoprevention or chemotherapy trials. Additionally, in another embodiment, the data collected is used as a prognostic indicator in patient follow up studies.

### Clinical Management of CaP

In recent years, prostate specific antigen (PSA) screening has led to an explosion in the number of patients with clinically localized CaP. Some prostate tumors are indolent and the patients may survive with it for many years without any treatment, whereas some tumors may be virulent and kill the patients in a few years. Unfortunately, there exists no good prognostic indicator that can predict the potential for CaP progression and metastasis. Thus, there is no clear-cut consensus on optimal treatment for men with clinically localized CaP. In 1995, the American Urological Association Guideline for the Management of Localized Prostate Cancer noted a lack of clinical trials and no compelling data on the superiority of one treatment over others for the disease. D'Amico et al introduced a stratification of CaP patients in a low risk group (combined Gleason score 6 or less, PSA less than 10 ng/ml and stage T2a or less) and in a high risk group (combined Gleason score greater than 7, PSA more than 10 ng/ml, or stage T2c or higher) that helped clinicians to classify patients based on risk factor. This, however, did not improve the clinical decision making process, as one-third of the patients who died of CaP are from the low-risk group at the time of their first diagnosis. Thus, dependable biomarkers as diagnostic and/or prognostic indicators is important in cancer, in general, and in the field of CaP, in particular. The clinical use of biomarkers for diagnosis and/or prognosis warrants a reproducible and reliable method of quantitation of fluorescence intensities of fluorescence-tagged biomarkers or immuno-fluorescence of immuno-histochemistry (IHC) assay slides.

### Quantitative Estimation of Tissue Biomarkers

An automated scoring system for assessing biomarker expression in tissue microarray (TMA) sections called the automated quantitative analysis (AQUA) system has been developed. The AQUA system is linked to a fluorescent microscope system that detects the expression of biomarker proteins by measuring the intensity of antibody-conjugated fluorophores within a specified subcellular compartment (typically including the nucleus, cytoplasm, and plasma membrane) within the tumor region of each tissue microarray spot or the selected areas of tissue sections. The result is a continuous score of immuno-fluorescence intensity for the tumor. An AQUA analysis removes the subjectivity of the traditional scoring system and provides more continuous and reproducible scoring of protein expression in tissue samples. It is also thought that using immuno-fluorescence based AQUA results in a marked increase in sensitivity of detection, as compared to standard IHC assay. An automated microscope stage and digital image acquisition in an AQUA system makes biomarker validation 30-50 times faster than the traditional pathologist based scoring. Biomarkers have been validated using AQUA system. Some of these biomarkers are now accepted as state-of-the-art in quantitation of cellular and subcellular levels of tissue biomarkers in IHC analysis. The AQUA system, however, can quantitate fluorescence intensities of proteins only in fixed cells and tissues. It cannot determine the levels, the real-time kinetics of production or intracellular translocation of small molecule biomarkers and metabolites, such as reactive oxygen species (ROS) that may regulate cellular oxidative stress, damage cellular macromolecules and act as subcellular signaling molecules leading to pathophysiology, including CaP occurrence, recurrence, inflammation and progression. Here, we use a BD Pathway Bioimager or equivalent instrument to quantitate the ROS levels in live cells and resected animal and human tissues to supplement the AQUA system in estimating small molecule metabolites, their production kinetics and their subcellular translocation.

### Oxidative Stress Induced Human Diseases

Approximately 1-4% of the oxygen that we breathe in is believed to be converted to ROS in the cells of our body. The mitochondrial electron transport chain is one of the leading generators of cellular ROS. We have demonstrated that under certain conditions, tissue specific oxidation of some small molecules, including polyamine catabolism in the prostate gland, contribute immensely to the cellular ROS pool. When cellular ROS production exceeds the cell's detoxification capacity and overwhelms the cellular repair system, oxidative damage occurs in cellular proteins, DNA, RNA and phospholipids. This damage disrupts mitochondrial oxidative phosphorylation and leads to significant cellular damage and cell death. ROS and cellular damage contributes to several human pathologies, including Parkinson's and Alzheimer's diseases, Friedreich ataxia, ischemia-reperfusion injury, diabetes and aging. In addition to these pathological roles, ROS can cause covalent modifications of DNA bases leading to gene mutation and also act as cell signaling molecules that may promote unregulated cell proliferation giving rise to hyperplasia or neoplasia. Several human diseases and malignancies may be induced by ROS, and the most compelling is the evidence of ROS induced CaP occurrence, recurrence and progression.

### ROS and CaP

ROS are produced in the prostate gland at a relatively higher level, as compared to that in most other organs. ROS affect prostate tissue by causing inflammation, altering cell growth, DNA base oxidation and mutagenesis, inducing apoptosis and modifying gene expression. The relatively higher levels of ROS and the subsequent chain of events are believed to initiate prostate carcinogenesis as well as CaP progression and metastasis. ROS are produced during lipid peroxidation and other metabolic oxidative activities and cause lipid modification. They also damage and mutate DNA and alter the activities of thiol-dependent enzymes. In addition, the relatively higher levels of ROS act as cell mitogens and the redox alterations play a key role in specific signal transduction pathways. High fat diets are likely to produce ROS due to high lipid peroxidation, which is commonly believed to be a cause of the relatively higher incidences of CaP in the industrialized nations, as compared to that in the developing countries. Some studies have shown a decrease in CaP incidence with the consumption of certain dietary anti-oxidants, such as β-carotene, β-lycopene, Vitamin E and selenium, most of which scavenge oxygen free radicals and thereby, reduce cellular ROS.

### ROS Induce Occurrence, Chronic Inflammation, Recurrence and Progression of CaP

In the past 5 years, considerable experimental results in laboratory animals and clinical data directly linked increased ROS with an increase in prostate carcinogenesis. Oberley et al used classical immuno-histochemistry methods to measure ROS-induced damage to protein and DNA in archival paraffin blocks of resected malignant and normal human prostate tissues. Malignant and metastatic human prostate tumor cells showed convincing evidence of markedly higher ROS-induced protein and DNA modifications than did the normal prostate tissue. Ho and coworkers demonstrated that oxidative damage in DNA and protein is significantly higher in the pre-neoplastic lesions such as prostatic intraepithelial neoplasia (PIN) as compared to that in the adjacent normal prostate tissues in the TRansgenic Adenocarcinoma of Mouse Prostate (TRAMP) tissues. These results indicate a key role of ROS in CaP occurrence and progression. Recent studies also show that intracellular ROS play a key role in androgen-independent growth, chronic inflammation and proliferation of ADT (Androgen Deprivation Therapy) treated androgen-dependent LNCaP human prostate cancer cells. Thus, it is evident that ROS are not only involved in CaP occurrence and recurrence, but also play a critical role in CaP progression to more virulent androgen-independent growth. In the last couple of years, there are accumulating evidence that activation of polyamine metabolic enzyme spermidine/spermine acetyl transferase (SSAT) is a key enzyme in responsible for the increase in ROS in the prostate tissue. In some embodiments, a reliable prognostic biomarker of CaP progression requires the determination of the levels of expression of protein biomarkers from an IHC assay and determination of the subcellular levels and distribution of small molecule metabolites such as ROS, as well as the real-time kinetics of their production in order to gather all information needed for a reliable prognosis of CaP and other cancers. In other embodiments, a method for quantitative estimation of intracellular small molecule levels in frozen tissues and their production kinetics in live cells (fresh tissues) and their subcellular distribution is required for determining efficacies of anti-oxidants or odidative stress modulator drugs used in chemoprevention clinical trials, and also for use as a prognostic or diagnostic biomarker.

### Chronic Non-Resolving Inflammation (CNRI) in Prostate, Breast and Certain Other Cancers

Chronic non-resolving inflammation contributes to cancer development including tumors not epidemiologically linked to inflammation. Inflammation is an essential element of the tumor micro environment and is also present in tumors not epidemiologically linked to inflammation. A leukocyte infiltrate and soluble inflammatory mediators such as cytokines, and chemokines contribute to cancer-related inflammation. Conditions predisposing to cancer (e.g., prostitis for inflammation associated prostate disease, hepatitis for HBV induced liver disease and hepatocellular carcinoma and for ulcerative colitis for colitis-associate cancer) or genetic events which underlie neoplastic transformation orchestrate the build-up of an inflammatory microenvironment. Therefore, an intrinsic pathway driven by oncogenes and an extrinsic pathway driven by chronic inflammatory conditions which contribute to cancer-related chronic inflammation. Inflammatory cells are involved in tumor invasion of vasculature and other tissues and metastasis. Mediators of inflammation including toxins and herbicides, like Dioxin (Agent Orange used in the Vietnam War as a defoliant). Agent Orange induces a high rate of production of hydrogen peroxide and pathophysiologocal ROS in the human prostate. Agent Orange is now commonly known as a major cause of prostate cancer in those men who were heavily exposed. Many of these Vietnam War Veterans who were heavily exposed to Agent Orange Dioxin can have increased prostate cancer and some have metastatic dissemination. It is now appearent that chronic inflammation of the prostate from bacteria of viral infections or exposures to herbicides, like Dioxins, can have chronic inflammation and inflammatory cells which contribute to local invasion and dissemination and can form metastatic sites. Inflammatory cells and reactive oxygen species mediators of cell and DNA damage are key elements in the involvement of specific glands (prostate), organs and tissues, including inflammatory breast and prostate cancers and their subsequent metastases, including metastasis to bone and nerves.

We have discovered that: (a) increased production rates of ROS, especially including superoxide and hydrogen peroxide. This can also occur in other solid tumors, for example in chronically inflamed liver, pancreas, prostate, colorectal or breast cells. High rates of production of hydrogen peroxide are responsible for oxidative DNA damage, including high 8-OHdG levels in human prostate carcinomas and also in human colorectal or breast carcinomas; (b) there is a general direct positive proportional correlation between Oxidative Stress levels, as measured by the proprietary dye oxidation assay described herein, as 8-OHdG levels and 8-OHdG-specific DNA modification activity, including, for example, but not by way of limitation, the 8-OHdG-specific lyase activity or hOGG1 expression in mouse and human tumors; (c) 8-OHdG levels can be maintained at a constant high level among early- and advanced-stage prostate or colorectal carcinomas in experimental mouse models of PCa or in primary human tumors from PCa patients, and this adjustment appears to be regulated in part by enzymes. For an example this can involve DNA damage repair enzymes, as for example with 8-OHdG-specific lyase activity or hOGG1 or other appropriate damage repair enzymes, often under transcriptional controls; and/or (d) no mutation may be detected, as for example in hOGG1 where gene mutations are not detected in patients with colorectal carcinomas. Polymorphism of this gene appears to be independent of 8-OHdG-specific lyase activity in colorectal carcinomas. Further understanding of tumor metabolism in persistent and high rates of production of hydrogen peroxide-mediated oxidative stress, chronic inflammation and cellular DNA damage, including oxidation of DNA (i.e. OH-8dG modification in oxidized prostate or colorectal cell DNA) has lead to our development of a new dye oxidation and SNP (Single Nucleotide Polymorphism) CHIP diagnostic, and can be a standalone or a companion diagnostic with a therapeutic and preventive drug strategy for prostate adenocarcinomas and other carcinoid tumors, including breast and colorectal carcinomas.

For a specific example in CaP, from 2001-2005, the median age at diagnosis for cancer of the prostate was 68 years of age, and in the US from 2001-2005, the median age at death for CaP was 80 years of age (http://seer.cancer.gov/csr). The incidence of CaP has also been found to be population specific. The age-adjusted death rate was 26.7 per 100,000 men per year, based on CaP patients who died in 2001-2005 in the US. Among white men, the CaP rate was 24.6 per 100,000 men; among black men the CaP rate was 59.4 per 100,000 men; among asian/Pacific Islanders, the CaP rate was 11.0 per 100,000 men; among American Indian/ Alaskan Natives, the CaP rate was 21.1 per 100,000 men; and among Hispanics, the CaP rate was 20.6 per 100,000 men. According to the National Cancer Institute, and based on CaP, rates from 2003-2005, 15.78% of men born today will be diagnosed with cancer of the prostate at some time during their lifetime. This number can also be expressed as 1 in 6 men will be diagnosed with CaP during their lifetime. These statistics are called the lifetime risk of developing cancer. Lifetime risks may also be given in terms of the probability of developing, or of dying from, cancer. Based on cancer rates from 2003 to 2005, it was estimated that men had about a 44 percent chance of developing cancer in their lifetimes, while women had about a 37 percent chance of developing cancer. When calculating the probability of developing CaP between two age groups, for example, 8.04% of men will develop cancer of the prostate between their 50th and 70th birthdays, as based information available on the world wide web at http://seer.cancer.gov/statfacts/html/prost.html. Prostate cancer is often a latent disease. Many men carry prostate cancer cells without overt signs of disease. The progression of the disease usually, goes from a well-defined mass within the prostate to a breakdown and invasion of the lateral margins of the prostate, followed by inflammation and metastasis to regional lymph nodes, and eventual metastasis to the bone marrow. Cancer metastasis to bone is common and often associated with uncontrollable pain. Autopsies of individuals dying of other causes show prostate cancer cells in 30% of men at age 50 and in 60% of men at age 80! Furthermore, prostate cancer can sometimes take up to 10 years to kill a patient after the initial diagnosis.

Current microarray technologies and the sequencing of the human genome have significantly enhanced the potential for investigations in all fields and particularly in the area of cancer, circulating tumor cells and inflammation diagnostics. High-throughput gene expression profiling technologies offer an opportunity to uncover critical molecular events in the development and progression of various cancers and can be used to design improved prognostic testing and effective treatment strategies. High-density tissue microarrays (TMA) are useful for profiling protein expression in a large number of samples (Rubin M. A. et al., Am J Surg Pathol. 2002 March; 26(3):312-9), and previous transcriptome analyses in CaP and various other malignancies have provided valuable information for the assessment of patient group classifications such as subgroups of patients that are likely to respond to a particular therapy (Sondak, V. K. Adjuvant therapy for melanoma. Cancer J7 Suppl 1, S24-7. (2001)). Particularly, in prostate cancer, microarray analysis provides a useful way to examine large numbers of clinical samples for prostate cancer biomarkers.

Prostate cancer is typically diagnosed with biopsy examination following a digital rectal exam and/or prostate specific antigen (PSA) screening. An elevated serum PSA level can indicate the presence of CaP. PSA is used as a screening marker for prostate cancer because it is secreted only by prostate cells. A healthy prostate will release a stable amount—typically below 4 nanograms per milliliter into the circulation, or a serum PSA reading of “4” or less—whereas cancer cells release escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound may be used to map the prostate and show suspicious areas. Biopsies of various sectors of the prostate are used to determine if prostate cancer is present. Treatment options depend on the stage, grade, inflammation, and other clinical variables of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed, most often with nerve sparing techniques in attempts to preserve potency and urinary functions, and radiation, applied through an external beam that directs the dose to the prostate from outside the body or via low-dose radioactive seeds that are implanted within the prostate to kill prostate cancer cells locally.

For more aggressive prostate cancers, anti-androgen hormone therapy is also used, alone or in conjunction with surgery or radiation. Hormone therapy also includes luteinizing hormone-releasing hormones (LHRH) analog drugs like Zoladex (Goserelin), which block the pituitary from producing hormones that stimulate testicular testosterone production, or by surgical removal of the testis, alone or in combination with chemical (anti-androgens) that block androgenic signaling.

While surgical and hormonal treatments are often initially effective for localized CaP, these ADT (Androgen Deprivation Therapy) treatments can cause increased prostate inflammation which usually morphs to CR-CaP as metastatic CaP advanced disease and advanced metastatic CR-CaP which unfortunately remains essentially incurable to this day. Androgen Deprivation Therapy (ADT), including Combined Androgen Blockade, or CAB, using a LHRH agonist and a anti-androgen drugs in combination, and is common therapy for progressing CaP.

The increased inflammation in ADT treatment of CS-CaP patients can lead to massive apoptosis of the androgen-dependent malignant tumor cells and temporary tumor regression, but elevated and chronic non-resolving inflammation with elevated production rates of Oxygen Free Radicals and sustained Oxidative Stress. In most cases, however, the ADT treated CS-CaP can become chronically inflamed and the prostate tumor re-emerges as CR-CaP and proliferates independent of androgen as a signal for growth, in some cases by opening up alternative paths for signaling growth, with some CR-CaP able to use the Androgen Receptor and co-stimulator mediated signaling pathways.

The advent of prostate specific antigen (PSA) screening has led to earlier detection of PCa and may have reduced PCa-associated fatalities. However, the impact of PSA screening on cancer-specific mortality is still unknown pending the results of prospective randomized screening studies (Etzioni et al., J. Natl. Cancer Inst., 91:1033, 1999; Maattanen et al., Br. J. Cancer 79:1210, 1999; Schroder et al., J. Natl. Cancer Inst., 90:1817, 1998). A major limitation of the serum PSA test is a lack of prostate cancer sensitivity and specificity, especially in the intermediate range of PSA detection (4-10 ng/ml). Elevated serum PSA levels are often detected in patients with non-malignant conditions, such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445, 1995). This has resulted in a surge of equivocal prostate needle biopsies (Epstein and Potter J. Urol., 166:402, 2001). Thus, development of blood circulating prostate cancer tumor cells (CTCs) and tissue biomarkers or additional automated and sensitive methods to detect a patient at risk for prostate cancer remains. Thus, there remains a critical umet medical need for determining those at elevated risk for CaP or susceptible to prostate cancer and chronic inflammation, early-stage prostate cancer prognosis, and early intervention with respect to those different men with high rates of production of hydrogen peroxide and elevated oxidative stress, diets and genetics.

For example, when analyzing prostate tumors have identified differences in gene expression between African-American and European-American men that show the existence of distinct tumor microenvironments with or without elevated oxidative stress and immunosuppression (the area that includes the tumor and the surrounding non-cancerous tissue) in these two patient groups.

Many of the genes that are differentially expressed between the tumors of African-American and European American men are related to the immune system. The results of these types of studies suggest that biological differences may, in part, underlie the disparity in prostate cancer survival rates observed between African-Americans and European-Americans as opposed to Asian-Americans and their respective diets and genetics.

Prostate cancer is the second leading cause of cancer-related death among all men in the United States. However, incidence and mortality rates for this disease vary substantially among geographic areas and ethnic groups. Most notably, African-American men in the U.S. have the highest risk of developing prostate cancer, and, due to the development of more aggressive disease or chronic inflammatory prostate neoplastic disease, they have more than twice the mortality rate observed for other racial and ethnic groups.

The data suggests that African-Americans and European-Americans may respond differently to immune modulator and/or anti-inflammatory drugs and vaccines. This includes small molecule anti-antrogenic and other oxidative stress and signal transduction-, or cytoplasmic oxidase or mitochondrial membrane electron transport chain enzyme modulatory and anti-inflammatory therapeutics currently under study for prostate cancer in our laboratories. Understanding the oxidized DNA biolophysical and immunological and inflammation differences that play a role in the development and progression of cancer among racial and ethnic groups may aid in the development of drug and vaccine and other therapies directed towards such differences. It is important to understand and monitor the significance in differences in gene expression in prostate tumors from African-American, Asian-American, Native-American or European-American men. These analyses when reviewed with respect to diets and genetics can reveal differential expression of certain genes that influence metabolism, immune responses, inflammation, DNA repair and the progression of cancer in the tumors of African-American. Asian-American, Native-American or European-American men. Genes with altered expression in prostate or other solid tumors from certain ethnic groups of men can include genes encoding chemokines, cytokines, hormones and their receptors. Chemokines are proteins released by cells to regulate both immune system function and cell migration. For example, two of these genes, CXCR4 and CCR7, have been linked to cancer metastasis and encode proteins that are commonly produced during inflammation and infection.

In addition, expression of a number of genes that are induced by a type of cytokine referred to as interferon can be elevated in the African-American prostate tumor tissues. Interferon is produced by cells in response to various pathogens, including exogenous viruses or endogenous viruses, perhaps including XMRV associated with prostate cancer and a human gamma-retrovirus found in human Pca tumors. This observations suggested the possibility that viral infections, including human XMRV or human Hepatitis B Virus (HBV) could be associated with the development of prostate or other tumors in men and may for example, have different effects in African-Americans and may be dependent on hormones (androgens and estrogens), the androgen and other steroid receptor AR activated or co-activators including oncogenes (i.e. Jun D), Viral Genes (HBC X Oncogene and X Oncoprotein) or hormone and AR-mediated XMRV co-activators, which are inhibited by anti-androgens, and which may differ in these different ethnic groups.

Expression of genes in non-cancerous prostate tissue from African-American and European-American men may related to inflammation and immune system depending on oxidative stress states and the content of hydrogen peroxide in the tumor microenvironment than in non-cancerous prostate tissue with differing sates of peroxide-induced oxidative stress. Gene expression profiles in prostate tumors from different men in various ethnic groups may contain changes associated hormone differences, inflammatory, signal transduction, mitochondrial and immune responses. Mechanisms that block the tumor-destroying ability of inflammatory or immune cells can be more or less prevalent in some ethic groups, for example African-Americans, or certain viruses are more common in certain men or ethnic groups, including HBV-induced HCC in men (7-fold higher in men than women with chronic inflammatory HBV-induced Hepatitis) or XMRV in the tumors of men with PCa.

Genes expressed at different levels in tumors from genetically different men and different ethnic populations may differentiate between tumors from high risk and low risk African-American, Asian-American, Native-American or European-American men. For example, the genes, PSPHL and CRYBB2 are more highly expressed in the prostate tumors of African-Americans compared with European-Americans. While little is known about the functions of the two genes, PSPHL is located in a chromosomal region related to advanced tumor stage in prostate cancer. PSPHL SNP chips can be used for inflammation, immune supression, oxidative stress and prostate cancer.

Prostate tumors from certain patients can differ in their immunological, inflammatory and oxidative stress, oxidase enzyme and metabolism from European-American or other patients. Thus, the invention described herein can monitor the OS-related, inflammation-related, immune-related and metabolic-enzyme related differences in the genetic profiles and SNP profiles indicating to predisposing high or low risk factors for prostate oxidative stress, inflammation, dysfunctional immunity, tumor progression, tumor invasion of vasculature, tumor angiogenesis and metastasis.

### Anti-Oxidant Drugs for CaP Chemoprevention

Because of the direct connection between ROS and CaP, reducing the relatively high ROS levels in the human prostate gland is now an accepted strategy for developing chemotherapeutic or chemopreventive drugs against the occurrence, recurrence and progression of CaP. As many CaP chemopreventive agents that are either in or are being considered for clinical trials are anti-oxidants, a quantitative method of determining tissue ROS levels is necessary for the initial estimation of the efficacy of these drugs.

### Chemoprevention Clinical Trial

Recent epidemiologic studies have identified a number of dietary components and micronutrients as prostate cancer preventive agents that may have antioxidant activity. For instance, multiple studies have shown a reduced risk of prostate cancer in men who consume cruciferous vegetables, including broccoli, cauliflower, Brussel sprouts and cabbage. These vegetables contain glucosinolates, of which the major bioactive metabolites studied in cancer prevention include Indole-3-carbinol (I³C) and its polymeric product 3,3′-Diindolylmethane (DIM). DIM induces cytochrome P450 (CYP) class of enzymes to a lesser extent than does I³C and therefore, may be a safer agent for clinical use. Induction of these phase II enzymes has also been implicated in altered estrogen metabolism in both men and women. While the effects of altered estrogen metabolism in prostate cancer are less clear, DIM has been shown to block androgen receptor activation with decreased localization of the androgen receptor to the nucleus and decreased binding of dihydrotestosterone (DHT) to the androgen receptor in human LNCaP prostate cancer cells in laboratory studies. These findings have been associated with inhibition of proliferation of prostate cancer cells and induction of apoptosis in vitro and in a mouse xenograft model of prostate cancer. Release of cytochrome C from mitochondria with resulting caspase activation has been described as a mechanism of DIM-induced apoptosis. Furthermore, downregulation of PSA has also been observed with DIM. Given this promising data, the feasibility of DIM administration and level of activity in prostate tissue in patients with prostate cancer is currently being assessed in a NCI-sponsored clinical trials through the Chemoprevention Consortium, from which tissues are available for analysis.

### Other Anti-Oxidants as Potential CaP Chemopreventives

Green tea, a popular beverage in various parts of the world, has also been observed to have cancer chemopreventive effects in many model systems. Epidemiologic studies have implied regular consumers of green and black tea may have a lower risk of prostate cancer. Consistent with these data, geographic areas with relatively high consumption of green tea (Japanese and Chinese populations) have the lowest incidence of prostate cancer in the world. The chemopreventive effects of green tea against tumorigenesis and tumor growth have been attributed to the biochemical and pharmacologic activities of its polyphenolic constituents, especially epigallocatechin-3-gallate (EGCG). Potent antioxidant properties have been identified with EGCG. Therefore, EGCG is another agent which has generated interest in prostate chemoprevention and may also be evaluated at the tissue level when administered in the neoadjuvant setting to patients with prostate cancer, who are scheduled to undergo prostatectomy.

We have been following this strategy to develop potential CaP chemopreventive agents. A method of quantitation of tissue ROS is extremely useful to determine the pharmacodynamics of new classes of chemopreventive anti-inflammatory anti-oxidants and osidative stress/signal transduction modulator drug candidates. The method of quantitative estimation of tissue ROS may be clinically validated for determining drug efficacies, as well as for use as a reliable prognostic biomarker of SNP for CaP occurrence, inflammation, recurrence, progression and metastasis.

### Methods of Determining ROS in Cells and Tissues

There are several methods of quantitation of total ROS in cultured cells. The most widely used method is the intracellular oxidation of dichlorofluorescein diacetate (DCFH-DA) dye with cellular ROS that produces highly fluorescent DCF. This method is routinely used to quantitate total ROS in cultured cells. With the help of a BD Pathway Bioimager or an equivalent instrument, we have standardized DCFH oxidation kinetics in LNCaP cells, which were used to determine relative changes in ROS concentrations with time in the cell nuclei and cytoplasms in LNCaP cells grown in the presence and absence of androgen. DCFH-DA, however, has been shown to be a substrate to cellular peroxidases that may artificially increase the cellular ROS levels, particularly in the cells' cytoplasms. To circumvent this problem, a hydroethidine dye (HEt) oxidation method is now used for subcellular ROS estimation. Like DCFH dye, hydroethidine dye (HEt) is also oxidized by cellular ROS to yield the highly fluorescent E⁺ ion that can be detected by the BD Pathway Instrumented Bioimager or with equivalent digital imaging microscopic systems with reagents used at sub-nanomolar concentrations for sensitive and quantative detection. We also observed that HEt can also be safely administered in live animals and living human tissues and cells under conditions for treatment, sacrifice, tissue extraction, processing and experimentally monitoring of in vivo ROS production in prostate, liver, spleen and kidney tissues of various different animals. These proprietary diagnostic method and diagnostic kits have now been standardized in our laboratory. In addition, we have also standardized conditions for HEt dye oxidation in ex vivo assays for resected animal and human tissues. In one embodiment is a method of using the BD Pathway Bioimager or an equivalent instrument to quantitate the sub-cellular concentrations, real-time kinetics of production and subcellular translocation of ROS in resected human prostate tissues using the ex vivo HEt dye oxidation assay.

In summary, excess and unregulated ROS produced in the prostate gland not only play a central role in the occurrence and recurrence of CaP, but also in regulating expression of genes essential for the androgen-independent survival and proliferation of CaP cells. Thus, ROS is being recognized as an important biomarker for cancer prognosis, particularly in the field of CaP. For this reason, several anti-oxidant drugs either are in or are being considered for CaP chemoprevention clinical trials in adjuvant and neo-adjuvant settings. These drugs are generally non-toxic and their dose-limiting toxicity (DLT) usually manifest only at a very high dose. Therefore, the dosing regimens for these drugs need to be determined from their pharmacodynamic effects, which require accurate estimation of tissue oxidative stress. At present, there exists no quantitative method in determining tissue oxidative stress in animal or human tissues. At present, there is also an unmet medical need for improved methods to determine which patients are at high risk for chronic inflammation and non-resolving chronic inflammation associated with fatal prostate, breast, pancreas, colorectal and other carcinomas of epitheloid origins.

### Scope

Described herein, in some embodiments, is a method of using the IHC combined with AQUA method to quantitate ROS producing enzyme and ROS induced DNA damages in resected archival fixed and frozen prostate tissue samples, respectively. Also described herein is an AQUA method for quantitatively determining the ROS induced HEt dye fluorescence and a BD Pathway Bioimager or equivalent quantitation method of the ROS levels from real-time kinetics of HEt dye oxidation, the subcellular distribution of ROS and the time-dependent translocation of ROS between subcellular compartments in freshly resected human tissues. In another embodiment, the method described herein is used to determine the efficacy of the anti-oxidant drugs currently undergoing neo-adjuvant clinical trials as chemopreventive agents to reduce oxidative stress in the prostate tissues. All data is archived. At the end of the study, these data are compared with patients' Gleason performance score and cancer recurrence data to also establish this method for use of tissue ROS concentrations and distribution as prognostic indicator. In another embodiment is a method for following the subcellular levels, distribution and translocation of any fluorescence tagged small molecule biomarker and metabolites for in situ assay for enzyme activity and cellular metabolism that may be deemed important for diagnostic/prognostic or companion therapeutic drug or vaccine purposes.

In a first aspect, is a methods of detecting the presence or absence of nucleic acid segments in a gene locus of a subject, wherein the presence or absence of the nucleic acid segments in the gene locus or loci indicates an altered risk of inflammation and/or cancer. In preferred embodiments, the cancer is prostate cancer or colorectal cancer or HCC.

In one embodiment, the presence or absence of the nucleic acid segment(s) in the gene locus/loci is detected in an African-American subject. In another embodiment, the absence of the nucleic acid segment indicates an increased risk of prostate cancer in the African-American subject.

In a further embodiment, the nucleic acid segment comprises 133 base pairs of exon 1 of human PSPHL mRNA encoded by GenBank Accession No. AJOO1 612 corresponding to SEQ ID NO: 1.

In one embodiment, the nucleic acid segment comprises SEQ ID NO: 2. In one embodiment, the nucleic acid segment comprises SEQ ID NO: 13.

In one embodiment, the nucleic acid segment comprises SEQ ID NO: 14.

In one embodiment, the nucleic acid segment comprises SEQ ID NO: 15.

In a further embodiment, the presence of the insertion allele of the PSPHL gene locus is correlated with the expression of the PSPHL gene product. In a related embodiment, the absence of the insertion allele of the PSPHL gene locus is correlated with the absence of the PSPHL gene product.

In another embodiment, the deletion allele is associated with the expression of a set of genes. In one embodiment, the subject is homozygous for a deletion in the PSPHL gene locus. In another embodiment, the subject is heterozygous for a deletion in the in the PSPHL gene locus. In a related embodiment, the homozygous deletion allele is associated with the expression of a set of genes. In another embodiment, the heterozygous deletion allele is associated with the expression of a set of genes or XMRV. In one embodiment, the expression of the PSPHL gene product is associated with the expression of a set of genes or HBV X Oncogene. In one embodiment, the expression of the PSPHL gene product is associated with the expression of a set of genes or oncogene, such as JunD Oncogene.

In another aspect, the invention features a method of determining the ancestry of a subject comprising detecting the presence or absence of a nucleic acid segment in the PSPHL gene locus of a sample subject population, wherein the presence or absence of the genetic variation/genetic variant indicates the ancestry of the subject.

In one embodiment, the presence or absence of a nucleic acid segment is indicative of African, e.g., African American, Asian-American, Native-American or European, e.g., European-American, ancestry. In another embodiment, the absence of the nucleic acid segment identifies the population as an African American subject. In one embodiment of any one of the above aspects, the method further comprises selecting subjects with an increased risk of developing chronic inflammatory prostate cancer. In a related embodiment, the method comprises obtaining a sample from the subjects.

In another aspect, the invention features a biomarker for chronic inflammatory prostate cancer in an African American subject comprising an insertion in the PSPHL gene locus, wherein the presence of the biomarker is correlated with a decreased risk of prostate cancer.

In one embodiment, the insertion encodes a nucleic acid comprising 133 base pairs of exon 1 of human PSPHL mRNA encoded by GenBank Accession No. AJOO1 612 corresponding to SEQ ID NO: 1.

In another embodiment, the insertion encodes a nucleic acid comprising SEQ ID NO: 2.

In another related embodiment, the absence of the biomarker is correlated with an increased risk of prostate cancer in the African American subject.

In still another embodiment, the presence of the insertion in the PSPHL gene locus is correlated with the expression of the PSPHL gene product.

In one embodiment, the insertion allele is associated with the expression of a set of genes. In another aspect, the invention features a method of identifying a subject at risk for developing prostate inflammation or cancer comprising detecting the presence or absence of a nucleic acid segment in the PSPHL gene locus of a subject to determine the genotype of the subject, wherein the absence of the nucleic acid segment in the gene locus indicates an increased risk of prostate inflammation, OS, and/or cancer.

In yet another aspect, the invention features a method of determining the prognosis of a patient with prostate cancer comprising: detecting the presence or absence of a nucleic acid segment in the PSPHL gene locus of a subject, wherein the absence of the variation determines the prognosis of a patient with prostate inflammation, OS and cancer.

In one embodiment, the prognosis determines the course of a chemopreventative or chemotherapeutic treatment.

In another embodiment of any one of the above aspects, the subject is homozygous for a deletion in the in the PSPHL gene locus. In still another embodiment of any one of the above aspects, the subject is heterozygous for a deletion in the in the PSPHL gene locus.

In a further embodiment of any one of the above aspects, the subject is selected from an African American or other population.

In another embodiment of any one of the above aspects, the absence of the nucleic acid segment indicates an increased risk of, or risk of recurrence of, prostate cancer. In still another embodiment of any one of the above aspects, the nucleic acid comprises 133 base pairs of exon 1 of human PSPHL mRNA encoded by GenBank Accession No. AJOO1 612 corresponding to SEQ ID NO: 1.

In another related embodiment of any one of the above aspects, the nucleic acid comprises SEQ ID NO: 2. In another embodiment of any one of the above aspects, the nucleic acid comprises SEQ ID NO: 13, SEQ ID NO; 14 or SEQ ID NO; 15.

In one embodiment of any one of the above aspects, the presence of the insertion allele of the PSPHL gene locus is correlated with the expression of the PSPHL gene product. In another embodiment of any one of the above aspects, the absence of the insertion allele of the PSPHL gene locus is correlated with the absence of the PSPHL gene product. In a related embodiment of any one of the above aspects, the homozygous deletion allele is associated with the expression of a set of genes. In another related embodiment of any one of the above aspects, the heterozygous deletion allele is associated with the expression of a set of genes.

In another embodiment of any one of the above aspects, the presence or absence of a nucleic acid segment in the PSPHL gene locus is determined using a polymerase chain reaction (PCR) assay. In a related embodiment, the PCR assay is a multiplexed PCR assay. In a further related embodiment, the PCR is carried out using primers comprising the nucleic acid sequences as set forth as SEQ ID NO: 3 and SEQ ID NO: 4 and primers comprising the nucleic acid sequences as set forth as SEQ ID NO: 5 and SEQ ID NO: 6. In another related embodiment, the nucleic acid sequences as set forth as SEQ ID NO: 3 and SEQ ID NO: 4 amplify a 133 base pair fragment of the insertion sequence in exon 1 of the PSPHL gene. In still another related embodiment, the nucleic acid sequences as set forth as SEQ ID NO: 5 and SEQ ID NO: 6 generate an amplicon only if the insertion sequence is absent.

In another embodiment of any one of the above aspects, the subject has previously been treated for prostate inflammation and/or cancer.

In still another embodiment of any one of the above aspects, the measurement is performed after surgery or therapy to treat prostate cancer.

In another aspect, the invention features an antibody to detect PSPHL protein in cells and tissues with PSPHL genotypes. In one embodiment, the antibody is polyclonal. In another embodiment, the antibody is monoclonal. In a related embodiment, the polyclonal antibody is directed to the 72AA antigen of prostate cells corresponding to SEQ ID NO: 7.

In another aspect, the invention features a kit for use in identifying a subject at risk for developing prostate cancer comprising primers directed to amplify a 133 base pair sequence of exon 1 of human PSPHL mRNA encoded by GenBank Accession No. AJOO1 612 corresponding to SEQ ID NO: 1, and instructions for use to detect inflammation and OS by proprietary dye oxidation assay.

In one embodiment, the primers comprise the nucleic acid sequences as set forth as SEQ ID NO: 3 and SEQ ID NO: 4.

In another embodiment, the primers comprise the nucleic acid sequences as set forth as SEQ ID NO: 5 and SEQ ID NO: 6.

In another aspect, the invention features a kit comprising primers comprising the nucleic acid sequences set forth as SEQ ID NO: 3 and SEQ ID NO: 4, and instructions for use.

In another aspect, the invention features a kit comprising primers comprising the nucleic acid sequences set forth as SEQ ID NO: 5 and SEQ ID NO: 6, and instructions for use.

In another further aspect, the invention features a kit comprising primers designed against the nucleic acid sequence set forth as SEQ ID NO: 17, and instructions for use.

In still another aspect, the invention features a kit comprising primers designed against the nucleic acid sequence set forth as SEQ ID NO: 18, and instructions for use.

In still another further aspect, the invention features a kit comprising primers designed against the nucleic acid sequence set forth as SEQ ID NO: 19, and instructions for use. In one embodiment of any one of the above aspects, the kits further comprise instructions for use in PCR assay. In one embodiment, the PCR is multiplexed PCR.

In another aspect, the invention features a kit for use in identifying a subject at risk for developing prostate cancer comprising: an antibody directed to a PSPHL antigen, and instructions for use. In another aspect, the invention features a kit comprising an antibody directed to a PSPHL antigen. In another aspect, the invention features a SNP Chip detection method, either automated or in a kit.

In one embodiment of any one of the above aspects, the antibody is monoclonal.

In one embodiment of any one of the above aspects, the antibody is polyclonal. In a related embodiment, the polyclonal antibody is used to detect the 72AA antigen. In another related embodiment, the polyclonal antibody is directed to a sequence encoded by SEQ ID NO: 7.

Genetic variation refers to heritable DNA level differences that exist in all living organisms. There are 3 billion chemical base pairs that make up human DNA. One of the most common types of genetic variation is called single nucleotide polymorphisms (SNPs). Each SNP accounts for only one base pair difference, however there are millions of SNPs that account for an average genetic difference between humans in about 0.08% of the 3 billion chemical base pairs in the human genome. Structural variation is another type of genetic variation that each involves at least 1000 such chemical base pair codes. These longer stretches of DNA can be deleted, duplicated, or inserted, leading to change in DNA dosage (therefore also termed copy number variation). They can also be inverted in orientation or translocated to a different location in the genome. In recent years, knowledge regarding this type of variation has increased, following the improvement of genomic technologies such as microarrays. These variations, by definition, are differences that can be inherited, not alterations such as the ERG-TMPRS S2 fusion that has been discovered in prostate cancer, which are not heritable. Genomic variants of all sizes and types can contribute to genetic disease. From the twin studies the understanding has emerged that there is a strong genetic basis for human prostate cancer. Many geneticists have made use of the genetic variations such as SNPs to identify the risk factors that would elevate the chance of developing prostate cancer if inherited. Indeed, they have been very successful. However, these studies are not without limitations. One such limitation is that the gene or genes that contribute to this elevated risk have not been definitively identified. Yet another limitation is that corresponding studies in African American men, the highest risk group, have not been conducted in similar scale with similar intensity. Yet another limitation is that XMRV genetics is still under examination. While commercial tests, some costing a few hundred dollars, are being developed to assess inflammation and prostate cancer risk, with the hope to benefit humankind by early diagnosis and timely treatment to reduce prostate inflammation and cancer mortality, it remains to be established whether these tests are equally effective in identifying high risk African American men, already a high risk group for developing prostate cancer. Further, the use of structural variation for identification of prostate cancer genes is a research area that has not been explored. Recently, novel genomic variations have been identified with the potential to address the above-mentioned aspects of prostate inflammation and cancer biology and immunology. Thea locus on chromosome 7, termed PSPHL, that harbors a segment of DNA that can be either present or missing from the human genome. When it is present, the PSPHL gene is expressed in the prostate and may function through the expressed products. When it is absent, the gene is not expressed in the prostate or any other tissues in the body because there is no genetic code to start with. Furthermore, it has been found that this segment of DNA is present in −96% of healthy African Americans, but deleted in most healthy Americans of European descent.

Genetic differences between humans is shaped by the common ancestry of humans in Africa some 50,000 years ago. Rare forms of genomic variations that seem to be skewed in its distribution among different populations are, in certain cases, more commonly associated with medically relevant traits. Many such genetic differences have been analyzed in detail. One clear example is the genes that confer resistance to malaria. Another example that may be applied to human prostate inflammation, immunity and cancer, is the use of ancestry informative markers to identify prostate inflammation, OS, and cancer genes in admixture studies.

The present invention also provides methods of detecting the presence or absence of a nucleic acid segment in the (Phosphoserine phosphatase-like) PSPHL gene locus of a subject, wherein the presence or absence of the nucleic acid segment in the gene locus indicates an altered risk of inflammation, OS or cancer.

In certain embodiments, the invention provides biomarkers for prostate inflammation, OS and cancer in an African American subject comprising an insertion in the PSPHL gene locus, wherein the presence of the biomarker is correlated with a decreased risk of prostate inflammation and cancer. Accordingly, the present invention presents a biomarker or biomarkers that are differentially present in samples of prostate cancer subjects and control subjects, or in subjects of different populations, or in subjects at different stages of cancer, e.g. prostate inflammation, cancer, progression, and the application of this discovery in methods and kits for determining the presence of chronically inflamed prostate cancer. These biomarkers are found in samples from prostate cancer subjects at levels that are different than the levels in samples from subjects in whom prostate inflammation, immunosupression or cancer is undetectable. Accordingly, the amount of the biomarker, Circulating Tumor Cell (CTC) in blood, or, one or more biomarkers, found in a test sample compared to a control, or the presence or absence of one or more markers in the test sample provides useful information regarding the inflammation, suppressed immunological or cancer status of the subject.

In accordance with one embodiment of the invention, a set of genes whose expression correlates with expression of the PSPHL gene product were identified. Further, in preferred embodiments, the homozygous deletion allele is associated with the expression of a set of genes. In other embodiments, the heterozygous deletion allele is associated with the expression of a set of genes. These genes may also be useful to determine disease onset/progression/inflammation, to determine prognosis, to determine risk of occurrence, inflammation, OS, immune suppression, recurrence and to determine course of therapy in subjects having routine health screenings, routine prostate inflammation or prostate cancer screenings, in those suspected of having prostate infection, inflammation or cancer, for those with known a risk of prostate cancer, for those previously treated for prostate cancer or with relatives with prostate disease. The genes described herein are also useful as novel therapeutic targets with or without companion diagnostics.

In one embodiment, the absence of the nucleic acid segment in the PSPHL gene product indicates an increased risk of prostate inflammation and cancer in an African American subject. The absence of the nucleic acid segments as described herein is useful, for example, to predict inflammation or disease progression. The claimed methods allow for earlier detection of disease occurrence, inflammation, recurrence/progression, metastasis and therefore earlier treatment of subjects with chronic inflammatory recurrent/progressive disease.

In addition, knowledge of genetic changes that occur in prostate inflammation, OS, and cancer enable the design and screening for targeted therapeutic agents that interact with the targets. The interaction may be direct or indirect. Therapeutic agents are agents that improve survival in subjects with disease, including advanced Chronic Non-Responsive Inflammatory CR-PCa disease. Provided herein are methods of detecting the presence or absence of a nucleic acid segment in the PSPHL gene locus of a subject, methods of identifying a subject at risk for developing CS-PCa or CR-PCa, methods of determining the prognosis of a patient with prostate cancer, inflammation, OS, biomarkers for prostate cancer, and microarray and/or SBP CHIP or quantitative PCR technologies or CGH to identify molecular and genetic defects associated with prostate inflammation, OS, cancer onset or progression, and to correlate the expression of the biomarkers with the presence or stage of disease, thus providing diagnostic and prognostic markers for this disease and/or short term survival of the patient. Such markers are useful clinically to determine preventative and therapeutic strategies for subjects and guide subject drug or vaccine treatments.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “set of genes” refers to the one or more genes. In certain embodiments, one or more genes is particularly expressed when the nucleic acid segment in the PSPHL gene locus is present. In other embodiments, one or more genes is particularly expressed when the nucleic acid segment in the PSPHL gene locus is present. The set of one or more genes expressed when the nucleic acid segment in the PSPHL gene locus is present may be overlapping, may be the same, or may be different (e.g. the set of genes may have one, two three or more genes in common). The “set of genes” may refer to genes whose expression level, alone or in combination with other genes, is correlated with cancer or prognosis of cancer, for example prostate cancer. The correlation may relate to either an increased or decreased expression of the gene. For example, the expression of the gene may be indicative of cancer, or lack of expression of the gene may be correlated with poor prognosis in a cancer patient.

The term “detect” refers to identifying the presence, absence or amount of an object or molecule.

The term “nucleic acid” or “nucleic acid segment” as used herein refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethyl guanine, 2-methyladenine, 2-methyl guanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. In preferred embodiments, the nucleic acid segment is part of the (Phosphoserine phosphatase-like) PSPHL gene locus of a subject. In further examples the nucleic acid segment comprises SEQ ID NO: 2.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length nRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation specifies the sequence or order of amino acids in a nascent polypeptide. In certain preferred embodiments, the gene is Phosphoserine phosphatase-like (PSPHL). The phrase “Phosphoserine phosphatase-like (PSPHL) gene locus” is meant to refer to a gene locus on chromosome 7 that harbors a segment of DNA that can be either present or missing from the human genome. When the gene locus is present, the PSPHL gene is expressed in the prostate. When the gene locus is absent, the gene is not expressed in the prostate. Human PSPHL mRNA is encoded by GenBank Accession No. AJ001612. In certain embodiments, the PSPHL gene locus contains a nucleic acid segment comprising 133 base pairs of exon 1 of human PSPHL mRNA whose presence or absence corresponds to PSPHL expression.

As used herein the phrase “prostate cancer” refers to cancers of the prostate tissue and/or other tissues of the male genitalia, or reproductive or urinary tracts. As used herein, the term “gene expression” refers to the process of converting genetic information encoded in a gene into RNA (e.g., mRNA, rRNA, tRNA, or snRNA) through “transcription” of the gene (i. e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The phrase “determining a prognosis” or “providing a prognosis” refers to determining or providing information regarding the impact of the presence of cancer, for example prostate inflammation, OS, and cancer, (e.g., as determined by the diagnostic methods of the present invention) on a subject's future health (e.g., expected morbidity or mortality, the likelihood of getting chronically inflamed or cancerous, the risk of metastasis).

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to microarray analysis (with Significance Analysis of Microarrays (SAM) software), SELDI and immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance (SPR), ellipsometry and atomic force microscopy (AFM).

“Detect” as used herein refers to identifying the presence, absence or amount of the object to be detected.

“Marker” or “biomarker” in the context of the present invention refer to a polypeptide (of a particular apparent molecular weight) or nucleic acid, which is differentially present in a sample taken from subjects having prostate inflammation and cancer, as compared to a comparable sample taken from control subjects (e.g., a person with a negative diagnosis or undetectable prostate cancer, normal or healthy subject). The term “biomarker” is used interchangeably with the term “marker.” The biomarkers are identified by, for example, molecular mass in Daltons, and include the masses centered around the identified molecular masses for each marker, affinity binding, nucleic acid detection, etc.

A marker can be a polypeptide, which is detected at a higher frequency or at a lower frequency in samples of unaffected tissue from prostate cancer subjects compared to samples of affected tissue from prostate cancer subjects.

A marker can be a polypeptide, which is detected at a higher frequency or at a lower frequency in samples of human unaffected tissue from prostate cancer subjects compared to samples of control subjects.

A marker can be a polypeptide, which is detected at a higher frequency or at a lower frequency in samples of human affected tissue from prostate cancer subjects compared to samples of control subjects.

A marker can be differentially present in terms of quantity, frequency or both. “Subject” as used herein refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

“At risk for cancer” refers to a subject with one or more risk factors for developing a specific cancer. Risk factors include, but are not limited to, ancestry, gender, age, genetic predisposition, environmental expose, previous incidents of cancer, preexisting non-cancer diseases, and lifestyle.

“Unaffected tissue,” as used herein refers to a tissue from a prostate cancer subject that is from a portion of tissue that does not have gross disease present, for example tissue that is about 1, 2, 5, 10, 20 or more cm from grossly diseased tissue.

A polypeptide is differentially present between two samples if the amount of the polypeptide or nucleic acid in one sample is statistically significantly different from the amount of the polypeptide or nucleic acid in the other sample. For example, a polypeptide or nucleic acid is differentially present between the two samples if it is present at least about 25%, at least about 50%, at least about 75%, at least about 100%, 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide or nucleic acid is differentially present between two sets of samples if the frequency of detecting the polypeptide or nucleic acid in the cancer subjects' samples is statistically significantly higher or lower than in the control samples. For example, a polypeptide or nucleic acid is differentially present between the two sets of samples if it is detected at least about 25%, at least about 50%, at least about 75%, at least about 100%, at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition, i.e., cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “diagnostic amount” of a marker refers to an amount of a marker in a subject's sample that is consistent with a diagnosis of cancer. A diagnostic amount can be either in absolute amount (e.g., μg/ml) or a relative amount (e.g., relative intensity of signals). A “control amount” of a marker can be any amount or a range of amount, which is to be compared against a test amount of a marker. For example, a control amount of a marker can be the amount of a marker in a person without cancer. A control amount can be either in absolute amount (e.g., μg/ml) or a relative amount (e.g., relative intensity of signals). As used herein, the term “sensitivity” is the percentage of subjects with a particular disease. For example, in the cancer group, the biomarkers of the invention have a sensitivity of about 80.0%-98.6%, and preferably a sensitivity of 85%, 87.5%, 90%, 92.5%, 95%, 97%, 98%, 99% or approaching 100%.

As used herein, the term “specificity” is the percentage of subjects correctly identified as having a particular disease i.e., normal or healthy subjects. For example, the specificity is calculated as the number of subjects with a particular disease as compared to non-cancer subjects (e.g., normal healthy subjects). The specificity of the assays described herein may range from about 80% to 100%. Preferably the specificity is about 90%, 95%, or 100%.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab″ and F(ab)″2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologies. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

### Prostate Cancer Markers

The present invention is based upon the discovery that the presence or absence of a nucleic acid segment in the PSPHL gene locus of a subject indicates an altered risk of cancer, in particular prostate cancer, and the application of this discovery in methods and kits for determining the risk of prostate cancer. Some of these markers are found at an elevated level and/or more frequently in samples from prostate cancer subjects compared to a control (e.g., subjects with diseases other than prostate cancer). Accordingly, this novel structural variation of the PSPHL locus that is tightly linked to gene expression and demonstrates unusual patterns of population differentiation provides useful information regarding probability of whether a subject being tested is at risk for prostate cancer, and has prognostic value. The invention further provides biomarkers that find use in the diagnosis and characterization (e.g. the determination of risk of developing) prostate inflammation and cancer.

### Detection

The invention provides methods of detecting the presence or absence of a nucleic acid segment in the (Phosphoserine phosphatase-like) PSPHL gene locus of a subject, where the presence or absence of the nucleic acid segment in the gene locus indicates an altered risk of cancer, for example prostate cancer.

Prostate cancer or chronic inflammatory cancer disproportionately affects certain, for example men of African descent. In certain embodiments of the invention, the presence or absence of the nucleic acid segment in the PSPHL gene locus is detected in an African American subject, where the absence of the nucleic acid segment indicates an increased risk of prostate cancer in the African American subject.

As described herein, the nucleic acid segment is in the PSPHL gene locus of a subject. In particular examples, the nucleic acid segment comprises 133 base pairs of exon 1 of human PSPHL mRNA encoded by GenBank Accession No. AJOO1 612 corresponding to SEQ ID NO: 1. In more particular examples, the nucleic acid segment comprises SEQ ID NO: 2. SEQ ID NO: 2 is set forth below:

| SEQ ID NO: 2 | |
|---|---|
| aagccacaggctccctggctggcgtcagctaaagtggctgttgggtgtccgcaggcttct | 61 |
| gcctggccgccgccgcctataagctaccaggaggagctttacgacttcccgtcctgcggg | 121 |
| aagtggcgggcacgatcgcaaggtagcgcagaagcttctcaatggccagcgccagctgca | 181 |
| gccccggcggcgcactcgcctcacctgagcctgggaggaaaattcttccaaggatgatct | 241 |
| cccactcagagctgaggaagcttttctactcagcagatgctgtgtgttttgatgttgaca | 301 |
| gcacggtcatcagtgaagaaggaatcggatgctttcattggatttggaggaaatgtgatc | 361 |
| aggcaacaagtcaaggataacgccaaatggtatatcactgattttgtagagctgctggga | 421 |
| gaaccggaagaataacatccattgtcatacagctccaaacaacttcagatgaatttttac | 481 |
| aagttacacagattgatactgtttgcttacaattgcctattacaacttgctataaaaagt | 541 |
| tggtacagatgatctgcactgtcaagtaaactacagttaggaatcctcaaagattggttt | 601 |
| gtttgtttttaactgtagttccagtattatatgatcactatcgatttcctggagagtttt | 661 |
| gtaatctgaattctttatgtatattcctagctatatttcatacaaagtgttttaagagtg | 721 |
| gagagtcaattaaacacctttactcttaggaatatagattcggcagccttcagtgaatat | 781 |
| tggtttttttccctttggtatgtcaataaaagtttatccatgtgtcagaaaaaaaaaaa | |

SEQ ID NO: 2 is the transcribed mRNA sequence. However, genomic sequences that are not transcribed can also be used to detect the presence of the insertion allele. For example, SEQ ID NO: 13, SEQ ID NO: 14 and SEQ ID NO: 15 as set forth below can be used to as markers of the presence of the insertion allele.

SEQ ID NO: 13
CAGTGCAGCTGAGATCAGACTTCCATGTGTAACTCCCACTACCCTACCAG
GATGCCTTTTCATAAAGGTAAGAAATGTAAATTTGGCCTTAATATACAAA
GTTGCCAGGGCAGCACTGGGTCAGTTCTACATATAGTACTTCTACGTTCA
TCAGCGGAAACTTTAAGGGAAGGTGAAAATGCTTCTAGAAGGCGACTGGA
CACCAGCGCCTTTGGGCTCTTCTTCTAAGGCCAATAGTGACCTAAATTAT
TGACTGACTGCTCCAATCAAGTGGGCAAAAGGGTACCAAGGCCGCCAACA
TCAGACAAATTCACTTGAGGGCCTACCTATGTGCTTTGAAAGACAAAACT
GCTGTTGTGAAGGACACTGTATTTCAGAAAAACATAATCATATTAACAAC
TAGTAACAATGTAAAATGCTGATGTGTTGAATGCTACTTTAGAAAAACAT
GTTAAAATCTACAAAAAAAAATTTATGATACAAAACTACGTTATCAATCA
TCTAGCTAGCTAACTATCTACAGACATGGTTTTCATTTCTGTTGCTCAGG
ATGGAAAGCAGTGGGATGATCATAGCTCACTGCAGCCTTGAGCTCCTGGC
CTCAAGTGATCCTCCTGCCTCCTAAGTAGCTGGGGCCACAGGTGGACACA
GTGACACCTGGGTTTTTTCTTTTGTAGAGACAGGGTTTCACTACACTGCC
CAGGCTGGTGTCAAACTTTGGAGTCTCGCTGTGTCACCCAGGCTGGGGTG
CAGTGGTGGGGATCTCGGCTCACTGCAACCTCTGCCACCTGGGTTCAAGC
GATTACTTCCTGTATCAGCCTCCCGAGTAGCTGGGACTACAGGCATGTGC
CACCACACCCGGCTAATTTTTGTATTTTTTGTAGAGAACTGGGTTTCACC
ATGTTGGCCAGGCTGGTCTCAAACTCCTGACCTCAGGTGATCCACCTGCC
TCGGCCTCCCAAAGTGCTGGGATTACAGTCATGAGCCGATGAACACTTTC
TTATGCTATTAAATGGCCTAACCCAGGCGGGTCGTGGTGGCTCACGCCTG
TAATCCCGAAAACTCGGATGGCCAAGGTAAGAAGATCACTTGAACCTAGG
AATTCCAAACTGGCCTGGGCAACATAGCGAGACTCCCATCCCTACAAAAG
ATACAAAAATTAGGCCTGGCGCACACCACGCTCGGCTAGTTTTTGTACCT
TTTGTAGAGACAGGGTTGCGTCATGTTGCCTAAGCCGGTCTCGAACTCCT
GAACCCAAGCCATCCATCCTCCCGCCTCGGCCTCCCAAAGTGCTGGAGAT
TACAGGGGCCCAGCCAGCCTCATGTTTTCCTTTTAAGCAGTCCCTTCCCT
GTTGCACACTTGGGTTAGTTTTCTTTTTAATTTTTTTAAAACAGGGGGTT
ACCTCAATCTCGCAGCCTGGAGTGCTGTGGTGGGATCACAGCTCATTGGA
GCCTTGAACCTTGGGGTTCAAGTAGCTGGGGGGCTGAGGTAGGACTACAG
AGATGGGGTCCCGCCATGTTGCCAGGCTGCTCTTGGCCTGAAGGGCATCT
CCCGCTGGCCGTGCCCGGACATAGTTTTCTATTTTTGACCGACATAAACA
CTGTGCTGAGTCGGTGTTTGTCAACACACAGGACCTGGCGGGGAGGTCGC
GGTTACCAGGCTCCACTCTAAGTAGAAGACTGCCCAGCTCCAAGCACTGT
ACCTCCCGGTGACGTCGCCGAACGCCCGCCCTGTGACGATACCTAAGGCC
CACCTTCATGACGCCGCCGAAGGCCCGCCCCTGTGACGCCGCCGGAGGCC
CGCCCCTCACGCGGAGCCAATCGGAACTCGAGGCGGGGCTGTTGGGTCTT
CGGGAGCGCGCATGCGCGGGGGGCCACAGGCTCCCTGGCTGGCGTCAGCT
AAAGTGGCTGTTGGGTGTCCGCAGGCTTCTGCCTGGCCGCCGCCGCCTAT
AAGCTACCAGGAGGAGCTTTACGACTTCCCGTCCTGCGGGAAGTGGCGGG
CACGATCGCAAGGTAGCGCAGAAGCTTCTCAATGGCCAGCGCCAGCTGCA
GCCCCGGCGGCGCACTCGCCTCACCTGAGCCTGGgtACGTGCAGCCCCAC
AACACCTTCCCCAGCCAGGGCCCGGGGACCCCGGGAGCGTCCCCCGCCAC
CTGGCGCCGCTCATACCTGGGCAAGGGTGGGACCCCACTGAGGCCCGCCA
CGCATTAGGGAGCTTGCACTTCCCGAGTTTTGACCTCTGACGGGCAGTTG
TAATAGCATTAAAGTTTTTGAAATTTTGTAGCGGGGGTAGAAGGGGCTTG
GAAAGGGAAGAAAACATCTTTTAAAATATAACGTTCCGGCCGGGCCCGGG
GGTTAACCCTTTAATCCACATTTGGGAGGCCAGGCAGGGGATTACGAGGT
AGGAGTTAAGACCACCTGGCCGCATGGGGAACCCTGTTTTACTAAA

SEQ ID NO: 13 contains exon 1 and is assembled from sequences gnl|ti|945553014, gnl|ti|949228349, gi|41830605|gb|AADD01081234.1, and gnl|t|954837275.

SEQ ID NO: 14
GAGATATAGATGGGGAGATTTTTAATTTAGTTTTTTATTTAAAATTGTGT
TTATAAGGAAAGAGATTATTATGTNTTTTGATAGAGAATTTGGGAGAGTT
TGTTTAATTTAGAGAGAGATGGGTTAGNAAGATTTATAGGGTTGGANTGT
GATTTANGAAAAGATTTAAGGTGTTTGGAGAATAATTTTTGGGGAGGAAT
TTTTGGAATAAAAAGAAAANTTTGGTAAAAANGGTGGATTTGGTTTTANA
TGAAATAAAAAAATTAACCGGATGTGGTTGCACACGCCCTGTATNCCCAG
CTACTCAGGAGGCTGAGGCACGAGAATCACTAGAACCCAGGAGGTGGAGG
TTGCAGTAAGCCAAGTTCGTGCCACTACCCTCCAGCCTGGGCAACAGAGT
AAGACTCCATCTAAAAAAAAAATGAAGAAGAAGAAATTAGTGTAGTGTGG
GAAGTGAAAAAAAAAAAAAGAAAAGGAAAAGAAAAATGATTGAATTCATG
AATACACTCTTATGTGGCCTGCACCGACTTTGACACAAATTAGATTGGCT
TAGTAGGCAAGGGTGGGATCTTTTCATAATTTTATTTGATGTCTAAAATA
CATTTATCTTTTTTTCTTATAGGAGGAAAATTCTTCCAAGGATGATCTCC
CACTCAGAGCTGAGGAAGCTTTTCTACTCAGCAGATGCTGTGTGTTTTGA
TGTTGACAGCACGGTCATCAGTGAAGAAGGAATCGGTGAGCTAGCCAAAA
TCTGTGGTGTAGAGGACGCGGTGTCAGAAATGTAGGGATAGCATTTATTC
ACTTTATGAAATGATAAAGAATTTTTTTTTTTTTTTTTTTTGAGACAGAG
TCTCACTCTATTGCCCAGACTGGAGTGCAGTGGCACAATCTTGGCTTACT
GCAACCTCCACCTCCCAGGTTCAAGCGATTATCCTGCCTCAGCCTCCTGA
GTAGCTGGGATTACAGGCGTGTGCAACCACATCCGGCTAATTTTTTTATT
TTTAGTAGAGATGGAGTTTCACCATGTTGGTCAGGCTGGTCTCAAACTCC
TGACCTCGTGATCCACCCACCTCAGCCTCCCAAAGTGCTGGGATTACAGG
CGTGAGCCACCATGCCCGGCCATGAAATGATAAAGAATTTCTAAAGGGTG
GCTGTTTTGGATGAAGTGCTGGACCCTGGCTATAAAAGATGAGCACTAGG
CTTTTTCTCTCACCCCTTAGAGTTGAATTTGATATATTGAGAACTNTGCT
ATCGCTCC NN

SEQ ID NO: 14 comprises a Trace sequence that has exon 2 (red underlined), and the underlined 3′ sequence overlaps with gi|148184701|gb|ABBA01007462.1|Homo sapiens CTG_{—}1103276812568, whole genome shotgun sequence.

SEQ ID NO: 15
CACCCACCTCAGCCTCCCAAAGTGCTGGGATTACAGGCGTGAGCCACCATGCCCGGCCATGAAATGATAAAGAATTT
CTAAAGAGTGGCTGTTTTGGATGAAGTGCTGGACCCTGGCTATGGAACATGAGCACTAGGCTTTTTCTCTCACCCCT
TAGAGTTGAATTTGATATATTCATTCATTTATATAAATATTTATGGCAAAAAAATATGGATAAGACATGTTCTTAGC
TTGATTAGGGGAATGATGACCATCACTAACCAAAGAAGATGGCCTAGGGGGCAGGGCTCAAGTGTCCAGTGGCTTTG
TGTCATGGTTGAAAGTGTGGCCTTAGTGGTTCCCTGGAAAGGTTCAGAGTCTCTTGGTGGATCCTGGGTCAGAGCCC
CTCTCTCCCTCCCTCCCTCCTGCCCTCCCACCTCCTGCCCTGCAGCTGGGCACCACCCTCTGCAGCCCCAGTCCCCC
AGTCATGCACCATGTCATTTTCTTTTTTTTTTTTTTTTCagGACGGAGTCTCGCTCTGTCACCAGGCTGGAGTGCAA
TGGTGCAATCTCGGCTCACTGCAACCTCCGCCTCCTGGGTTCAAGCAATTCTCCTGCCTCCACCTCCTGAGTAGCTG
AAACTACAGgcACGCACCACCACACCCGGCTTATTTTTGTATTTTTAGTAGAGATGGTGTTTCACCATGTTGGCCAG
CCTGGTCTTGAACTCCTGACCTCGTGATCTGCCGGCCTCGGCCTCCCAAAGTGCTGGGATTACAGGCATGAGCCACC
ACACTCGGCCACCATTTTATTTTCAACTCCCTTTCATGGAAGAACGTTTAGCCTTTGGTCTCTTTCTTGATTTATGA
CAGCTCGCGGCTTTCAAGAAAACTACCTATGAATAGGCTGTGTAACTTTTATTTATTTATTTATTTTTTGAGATGGA
GTCTCTGTCTTCTAGGCTGGGGTGCAGTGGCATGATCTCGGCTCACTGCAACCTCCCCCTCCCAGGTTCCAGCAATT
CTCCTGCCTCAGCTTCCCAAGTAGCTGGGATTACAGGCATGCACCACCACACCTGGCTAATTTTTGCATTTTTTTTT
TATTAGAGATGGGGTTTTGTCATGTTGGCCAGGCTGGTCTCGAACTCCTGGCTTCAGATGAGCTGCCCACCTCAGCT
TCCCAAAGTTTTGGCATTATAGGCATGAGTCACTGCACCCAGCTTGTTAATGTTATTTTCAAGCACACCTTCAAAAG
TTTATTCCAAGGCCTTGCTCTCATAGCAGCAAAGCCTGTCTGTTGCATAGTGGGGCTCTTGTTAGCATCTTCCTTTT
GGTGGATGTTGTGTAGACCCGGGGCAGTAAGGCATGTTAACCTCGAGGACATTGGACCTGGCTGGCCTGACTGTTGG
GTCTCCCTCCTAGGACATGGCGAGCCATGGGTGGGGCAGTGTCTTTCAAAGCTGCTCTCACGGAGCACTTAGCCCCA
ATCCAGCCCTCCAGGGAGCAGGTGCAGAGACTCATAGCAGAGCACCCCCCACACCTGACCCCCAGCATAAGTAAGAG
GAGCCGCTGCTCCAGGTGTATTTCAGCACCAGTGTTGGGGAGCATGTCCTCCTGAGAGCATCTAACGATTGCTTTAG
AGAGCCCTCTGGGTGGTTTATTTATTTTCATTTTTATTTTTTAATTTTATTATTTTAAAAATATTTATTTATTTATT
TATTTATTTATTTATTTATTTATTTATTGAGGCAGAGTCTCGCTCTGTCGCCCAGACTGGAGTGCAATGGTACGATA
TCGGCTCACTGCAACTTCTGCCTCCCGGGTTCAAGTGATTCTCCTGCCTCAGCCTCCTGAGTAGTTGGGATTTCAGG
CACCCACCATGCCAGGCTAGTTTTTGTATTTTTAGTAGAGACAGGATTTCACCATGTTGTTTAGGCTGGTCTCGAAC
TCCTGACCTCAGGTGGTCTCCCCGCCTCAGACTCCCAAAGTGCTGGGATCACAGGCGTGAGCCACTGCGCCTGGCAA
AATTTTATTTATTTATTTTTTTTGAGACAAAGTCTCACTCTGTCGCCCAGGCTGGAGTGCAGTGGTGCAATCTCAAC
TCACTGCAACTTTTGCCTCCCAGGTTCACACGATTCTTCTGCCTCAGCCTCAGGAGTAGCTGGGATTACAGGCGCCT
GTCACCACACCTGGCTAATTTTTGTATTTTTAGTAGAGATGGGTTTCGCCATGTTGGCCAGGCTGGTCTCGAACTCT
TCATCTCAAGTGATCCACCTGCCTCGGCCTCCCAACGTGCTGAGATTACAGGTGTGAGTCACCACACCTGGCTTATT
ATTTTTTTTTTTAGATTCAGAGTCTCATTCTGTTGCCCAGGTTGGAGTACAGTGGTATAATCATGGCCCACTGCAGC
CTCGAACTCCTGGGGTCAAGTAGCCCTCCCACCTTAGCCTCCTTAGTAGTAGGGACTCCAGGTGAATGCCACCGTGC
CCAGCTAATTAAAGAATTTTTTTTTTTTTCTGAAAGTCTTGCTTTGTCACCCAGGCTGGAGTGCAGTGGCATGAACA
CAGCTCACTGCAGCATCAATCTTCTGGGCTCAAGGGATCCTCCTGCCTCTGCTTCCTAAGTAGCTGGGACTACAGAC
ATAGGCCACCATGCCAATTTTTTTTTTTTTTTTTTGAGATGGAGTCTCGCTCTGTTGCCCAGGCTGGAGTGCAGTGG
CGCCATCTTGGCTCACTGCAAGCTCCACCTCCTGGGTTCACGCCATTCTCTTCCCTCAGCCTCCCGAGTAGCTGGGA
CTACAGGCGCCTGCCACCACACCTGACTAATTTTTTGTATTTTTAATAGAGACGGGGTTTCACCATGTTAGCCAGGA
TGGTCTCAATCTCCTCACCTTGTGATCCACCTGCCTCAGCCTCTCAAAGTGCTGGGATTACAGGCGTGAGTCACCGC
GCCTGGCCCACCATGCCTAATTTTAAAAAAAAAATTTTTTTATAGACATAGCATCTCACTACATTGCCCAGGCTGGT
CTTGAACTTGTGGGCTTAAGTGACCAATTTTCACAACTCAGCCTCCCAAAGTGTTGGAATTATAGGTTTAAGACACT
ATACCCAGCCTTTAAGAATCTTCTTGTAGAGATGGAATCTTGCTGTGTTGCCCAGGCTGTTCTCAAGCTCCTTGCCT
CAAGTGATTTTCTACCTTGGCCTCCCAAAGTGCTGGAATTAAAGGCATGGACTGTGTGGTTCTTCTGGCACTTACAC
GTGGTCTTGTCTGGCCGGTTGTCTGGTCCTGTCTGTTTCTGCCTTTCCTCTTTCTCCAGGGAAAACCTAAGCTTTCC
TTTTTGTCCTCATCTTGTGTTTTTCTGGGTCCATGGGCAGAGTAGAGTTCTAGAATGGTTTCCTAAAGCAGCCAAGC
CCTACCCTTTGATTTCTAAGTACATTTAGAAAAACACATATAAGGCTGGGCATGGTGGCTCACGCCTGTAATCCCAG
CACTTTGCGAGGCCGAAGTGGATGGATCACCAGGTCAGGAGTTCGAGACCAGGCTAACCAACATGATGAAACCCTGT
CTCTACTAAAAATACAAAAATAAGCCGGGCACGGTAGCTCACGTCTGTAATCCCAGCACTTTGGGAGGCCAAGATGG
GTGTATCACCTGAGGTCAGGAGTTCGAGACCAGCCTGGCCAACATGGTGAAACCCAGTCTCTACTAAAAATACAAAA
AAAGTAGCTGGGCGTGGTGATAGGCGCCTGTAATCCCAGCTACCCAGGAGGCTGAGGGAGGAAAATCACTGGAACCC
GGGAGGCAGAGGTTGCTGTGAGCCGAGATCATGCCAGTGCACTCCAGCTAGGGCAACAGAGCAAGATTCCATCTCAA
AAAAAAAAAAAAAAAAAATTAGCCGGGCATTGTGGCATGGGCCTATAATACTCAGGAGGCTGAGGCAGGAGAAGCGC
TTGAACCCGGGAGGTAGAGGTTGCAGTGAGCCAAGATTGCAGCCTTGCACTCCAGCCTGGGTGACAAGAGTGAAACT
CTGTCTCAAAAAATAAAGAGAAAAACATATAGAAAACATTAACACCCCAGGCAGTATACCTTGTCAAACATACCTCA
GGCAAATGCATTCAGGAGAAGAAAATACATCTTATTTCCCTCTTCATGTTTCGTTTTTTTTTTTTGTTTTCTTTTGT
ATTACTCAGTGTTGGGTATTTTGTATTTTATTTTGCAGGGAGCTGGTAAGTCGCCTACAGGAGCGAAATGTTCAGGT
TTTCCTAATATCTGGTGGCTTTAGGAGTATTATAGAGCATGTTGCTTCAAAGCTCAATATCCCAGCAACCAATGTAT
TTGCCAGTAGGCTGAAATTCTACCTTAATGGTAAGATGTTAACGGTAACATGTTCCCTTTCTTAGCAGTTCCATTAT
TCAGTATTCTGGGTAATGTCTTTTGGAATGCAACTTGAACAGTCACATCAGAGTTAAATATTGAGATGAATGGTTCT
CTTTTTTTTTTTTTTTTTTTTTTTGAGACGGAGTCTCGCTCTGTCGCCCAGGCTGGGGTGCAGTGGCGCGATCTCGG
CTCACTGCAAGCTCCCCCTCCCGGGTTCACGCCATTCTCCTGCCTCAGCCTCCCCAGTAGCTGGGACTACAGGCGCC
CGCTACCATGTCAGGATAATTTTTTGTATTTTTAGTAGAGACGGGGTTTCTCCGTGTTAGCCAGGATGGTGTCGATC
TCCTGACCTCGTGATCCGCCCGCCTCGGCCTCACAAAGTGCTGGGATTACAGGCGTGAGCCACCGCGCCCGGCCGAG
ATGAATGGTTCTCTTAATTGATGTCTTTTGCCCTTTGGTACCCTTTGCTCAGCAAAACAGACTTAGATCATCACCTG
TCTTAGCTTTATTATCTATAACATCACCTGTCTGTACATAAATGTCTGCATTTCG

SEQ ID NO: 15 contains exon 3 (nucleotides 504-625) and 3′ sequence. The underlined sequence overlaps with the 3′ end of gnl|ti|226793227 (SEQ ID NO: 14). The presence of the insertion sequence can be detected by PCR of any of these sequences as set forth herein. In certain cases, the absence of the insertion sequence can be detected by absence of signal. In other cases, the absence of the insertion sequence can be detected by the presence of the deletion allele.

In still other cases, the presence of the deletion allele can be detected by PCR. Primers can be designed to the following exemplary sequences:

SEQ ID NO: 16
GTGGGCTCAGCCATCCTCCCAACTGAGCCTCCTGAGTAGCTGGGACTACA
GGTGTGAGCCATCACACTCAACTGGTGTAGCTATTTTAGAAGACAAACTG
GCAGTTTCTCAAAAGGCTAAACATACAGTCATCATATAATGCAACAATTT
CACTCCTAGGCATATATCCCAGAGAAATAGAAATATATGTCCACACAAAA
ACTTGTACAGCAATCTTCATAGCAGCATTGTTCATAATAGCCAATACGTG
GAAACAACCCAAATGTCCATCAACTGATGAACAGATAAACAAAATGCAGT
GTGTCTCTACCATGGAATATTATTCAGCCACAGAAGAAATGAAATACTGA
TACACACTATGACATAAAGGAACTTTGAAAACATTGTGCTAAGAGGGAAA
AAAAAGCCA
SEQ ID NO: 17
GTGGGCTCaAGCCATCCTCCCAACTGAGCCTCCTGAGTAGCTGGGACTAC
AGGTGTGAGCCATCACACTCAACTGGTGTAGCTATTTTAGAAGACAAACT
GGCAGTTTCTCAAAAGGCTAAACATACAGTCATCATATAATGCAACAATT
TCACTCCTAGGCATATATCCCAGAGAAATAGAAATATATGTCCACACAAA
AACTTGTACAGCAATCTTCATAGCAGCATTGTTCgTAATAGCCAATACGT
GGAAACAACCCAAATGTCCATCAACTGATGAACAGATAAACAAAATGCAG
TGTGTCTCTACCATGGAATATTATTCAGCCACAGAAGAAATGAAATACTG
ATACACACTATGACATAAAGGAACTTTGAAAACATTGTGCTAAGAGGGAA
AAAAAAGCCA
SEQ ID NO: 18
GTGGGCTCaAGCCATCCTCCCAACTGAGCCTCCTGAGTAGCTGGGACTAC
AGGTGTGAGCCATCACACcCAACTGGTGTAGCTATTTTAGAAGACAAACT
G GCAGTTTCT CAAAAGGCTA AACATACAGT CATCATATAA
TGCAACAATTTCACTCCTAGGCATATATCCCAGAGAAATAGAAATATATG
TCCACACAAAAACTTaTACAGCAATCTTCATAGCAGCATTaTTCATAATA
GCCAATACGTGGAAACAACCCAAATGTCCA TCAACTGATG
AACAGATAAA CAAAATGCAGTGTGTCTCTA CCATGGAATA
TTATTCAGCC ACAGAAGAAA TGAAATACTGATACACACTA
TGACATAAAG GAACTTTGAA AACATTGTGC
TAAGAGGGgAAAAAAAGCCA

Methods of the invention for determining the prostate cancer status, or the risk of developing prostate cancer of a subject, include for example, obtaining a biomarker profile from a sample taken from the subject; and comparing the subject's biomarker profile to a reference biomarker profile obtained from a reference population, wherein the comparison is capable of classifying the subject as belonging to or not belonging to the reference population; wherein the subject's biomarker profile and the reference biomarker profile comprise one or more markers as described herein.

The method may further comprise repeating the method at least once, wherein the subject's biomarker profile is obtained from a separate sample taken each time the method is repeated. Samples from the subject may be taken at any time, for example, the samples may be taken 24 hours apart or any other time determined useful.

Such comparisons of the biomarker profiles can determine prostate cancer status or risk of prostate cancer in the subject with an accuracy of at least about 60%, 70%, 80%, 90%, 95%, and approaching 100% as shown in the examples which follow.

The reference biomarker profile can be obtained from a population comprising a single subject, at least two subjects, at least 20 subjects or more. The number of subjects will depend, in part, on the number of available subjects, and the power of the statistical analysis necessary. The invention includes methods of qualifying prostate cancer status in a subject comprising:
(a) measuring at least one biomarker in a sample from the subject, and
(b) correlating the measurement with prostate cancer status.

The method may also comprise the step of measuring the at least one biomarker after subject management.

In a preferred embodiment, any one of the markers described herein or contemplated by the instant invention are used to make a correlation with the presence or absence of prostate cancer, wherein the prostate cancer may be any type or subtype of prostate cancer.

In another example, the biomarker is an insertion sequence corresponding to a nucleic acid segment in the PSPHL gene locus.

In another example, the biomarker is an insertion sequence set forth in SEQ ID NO: 2. Optionally, the methods of the invention may further comprise generating data on immobilized subject samples on a biochip, by subjecting the biochip to laser ionization and detecting intensity of signal for mass/charge ratio; and transforming the data into computer readable form; and executing an algorithm that classifies the data according to user input parameters, for detecting signals that represent biomarkers present in prostate cancer subjects and are lacking in non-prostate cancer subject controls. In some embodiments, the present invention provides methods for detection of the presence or absence of the nucleic acid segment in the PSPHL gene locus as described herein, wherein the absence of the sequence is associated with prostate cancer. In some embodiments, the presence or absence of the nucleic acid segment is detected in tissue samples (e.g., biopsy tissue). In other embodiments, detection is carried out in bodily fluids (e.g., including but not limited to, plasma, serum, whole blood, mucus, and urine). Exemplary methods are described below.

### Direct Sequencing Assays

In some embodiments of the present invention, a nucleic acid segment, for example, but not only limited to a nucleic acid segment in the PSPHL gene locus, is detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the insertion, the region containing the SNP) is sequenced using any suitable method, including but not limited to manual sequencing using radioactive marker nucleotides, and automated sequencing. The results of the sequencing are displayed using any suitable method. The sequence is examined and the presence or absence of a given SNP is determined.

### PCR Assay

In some embodiments of the present invention, the presence or absence of a nucleic acid segment is detected using a PCR-based assay. In some embodiments, the PCR assay comprises the use of oligonucleotide primers that hybridize only to the insertion or deletion allele (e.g., to the region of polymorphism). Both sets of primers are used to amplify a sample of DNA. In certain embodiments, the subject is homozygous for a deletion in the PSPHL gene locus. In other embodiments, the subject is heterozygous for a deletion in the in the PSPHL gene locus.

### Hybridization Assays

In preferred embodiments of the present invention, the presence or absence of a nucleic acid segment is detected using a hybridization assay. In a hybridization assay, the presence of absence of a given SNP is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided herein.

(1) Direct Detection of Hybridization. In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

(2) Detection of Hybridization Using “DNA Chip” Assays. In some embodiments of the present invention, the nucleic acid segment is detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which is incorporated herein by reference). Through the use of microelectronics, Nanogen's technology enables the active movement'and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is incorporated herein by reference). Protogene's technology was based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

### Enzymatic Detection of Hybridization

In some embodiments, hybridization of a bound probe is detected using a TaqMan assay (PE Biosystems, Foster City, Calif; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the

AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter. In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin). Numerous other assays are known in the art.

### Other Detection Assays

Additional detection assays that are suitable for use in the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); ESTVADER assay, Third Wave Technologies; See e.g., U.S. Pat. Nos. 5,846,717, 6,090,543; 6,001,567; 5,985,557; and 5,994,069; each of which is herein incorporated by reference; cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety).

### Mass Spectroscopy Assay

In some embodiments, a MassARRAY system (Sequenom, San Diego, Calif.) is used to detect the presence or absence of the nucleic acid segments as described herein. U.S. Pat. Nos. 6,043,031 ; 5,777,324; and 5,605,798; each of which is herein incorporated by reference, described Mass Spectroscopy assay. DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

### Data Analysis

In some embodiments, a computer-based analysis program is used to translate the raw data generated by the detection assay (e.g., the presence, absence, or amount of a given nucleic acid segment) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject. The present invention contemplates any method capable of receiving, processing, and transmitting the information to and from laboratories conducting the assays, information provides, medical personal, and subjects For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present or the subtype of cancer) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### Antibodies

Antibodies are well known to those of ordinary skill in the science of immunology. As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv) and fusion polypeptides. In one embodiment, an antibody that binds PSPHL polypeptide (e.g., PSPHL or a PSPHL variant) is monoclonal. Alternatively, the anti-PSPHL antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′.” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing PSPHL gene product (e.g. polypeptides gene product) or immunogenic fragments thereof, as an immunogen. In embodiments, a synthetic PSPHL protein sequence is used to generate the PSPHL antibody, In other embodiments, said sequence correspond to SEQ ID NO: 7 (MASASCSPGGALASPEPGRKILPRMISHSELRKLFYSADA VCFDVDSTVISEEGIGCF HWIWRKCDQATSQG).

One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a PSPHL polypeptide, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a PSPH: polypeptide, or immunogenic fragments thereof, can be expressed in cells in vitro, followed by isolation of the receptor and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Kits

The invention features kits for use in identifying a subject at risk for developing prostate cancer. In preferred examples, the kits for use in identifying a subject at risk for developing prostate cancer comprise primers directed to amplify a 133 base pair sequence of exon 1 of human PSPHL mRNA encoded by GenBank Accession No. AJOO1 612 corresponding to SEQ ID NO: 1, and instructions for use.

In certain embodiments, the invention features kits for the detection of AAIns or PSPHL. For example, a kit for detecting AAIns might include reagents for genomic DNA extraction, PCR reagents, and AAIns specific primers. A kit for detecting PSPHL gene expression might include reagents for mRNA isolation, RT-PCR reagents and PSPHL specific primers. A kit for detection of PSPHL protein expression may include primary antibodies against the PSPHL antigen coupled with general detection methods for specific binding.

In preferred embodiments, the kits of the invention feature primers for use in detecting a nucleic acid segment in the (Phosphoserine phosphatase-like) PSPHL gene locus of a subject. Thus, in certain examples, the kits preferably comprise primers. For example, the primers, in certain embodiments, comprise the nucleic acid sequences as set forth as SEQ ID NO: 3 and SEQ ID NO: 4. In other embodiment, the primers comprise the nucleic acid sequences as set forth as SEQ ID NO: 5 and SEQ ID NO: 6. In other preferred embodiment, the kits comprise the nucleic acid sequences set forth as SEQ ID NO: 3 and SEQ ID NO: 4, and instructions for use or the kits comprise the nucleic acid sequences set forth as SEQ ID NO: 5 and SEQ ID NO: 6, and instructions for use. The kits may further comprise instructions for use in PCR assay, for example in multiplexed PCR. Other kits that are featured in the invention are kits for use in identifying a subject at risk for developing prostate cancer comprising an antibody directed to a PSPHL antigen, and instructions for use. The antibody may be monoclonal or polyclonal. The polyclonal antibody may be used to detect the 72AA antigen, for example the polyclonal antibody comprising a sequence encoded by SEQ ID NO: 7. Tissue sources for the detection of the AAIns genomic DNA, the expressed products including mRNA and protein, may include any tissue sources where genomic DNA, mRNA, or protein can be retrieved.

It is possible in certain embodiments that the kits of this invention could include a solid substrate having a hydrophobic function, such as a protein biochip (e.g., a Ciphergen ProteinChip array) and a buffer for washing the substrate, as well as instructions providing a protocol to measure the biomarkers of this invention on the chip and to use these measurements to diagnose prostate cancer.

In one aspect, the invention provides kits for detecting a biomarker for prostate cancer in an African American subject. The invention provides kits for detecting the presence (or absence) of a nucleic acid segment in the (Phosphoserine phosphatase-like) PSPHL gene locus of a subject, wherein the presence or absence of the nucleic acid segment in the gene locus indicates an altered risk of cancer The kits include PCR primers for at least one marker, preferably the nucleic acid comprising SEQ ID NO: 2 as described herein, however, the kit may include identification of more than one biomarker as described herein. The kit may further include instructions for use and correlation of the biomarker with disease status. The kit may also include a DNA array containing the complement of one or more of the biomarkers, reagents, and/or enzymes for amplifying or isolating sample DNA. The kits may include reagents for PCR, for example, probes and/or primers, and enzymes. The kits of the invention have many applications. For example, the kits can be used to differentiate if a subject has prostate cancer or does not have prostate cancer (a negative diagnosis).

In one embodiment, a kit comprises: (a) a substrate comprising an adsorbent thereon, wherein the adsorbent is suitable for binding a biomarker, and (b) instructions to detect the marker or markers by contacting a sample with the adsorbent and detecting the biomarker or markers retained by the adsorbent. In some embodiments, the kit may comprise an eluant (as an alternative or in combination with instructions) or instructions for making an eluant, wherein the combination of the adsorbent and the eluant allows detection of the biomarkers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe, which is removably insertable into a gas phase ion spectrometer. In other embodiments, the kit may comprise a single substrate, which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

In another embodiment, a kit comprises (a) an antibody that specifically binds to a biomarker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a biomarker detected in a sample is consistent with a diagnosis of prostate cancer.

Reference cells may be normal cells (cells that are not prostate cancer cells) or prostate cells at a different stage from the prostate cancer cells being compared to. The reference cells may be primary cultured cells, fresh blood cells, established cell lines or other cells determined to be appropriate to one of skill in the art.

This invention should not be construed as limiting. All documents mentioned herein are incorporated herein by reference.

Other embodiments from the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### EXAMPLES

### Experimental Design and Methods

All experiments are performed in human tissues and cells collected following Institutional Review Board (IRB) approved procedures (see below). All patient data is collected and archived following HIPPA regulations.

### Study Design:

The data is collected using Immuno Histochemistry (IHC) method coupled with AQUA data analysis and quantitation. Two key components—SSAT enzyme levels and the amount of ⁸OH-Guanine levels in the DNA are used as indicator of oxidative stress levels in the prostate gland. We have previously established that the induction of SSAT enzyme leading to oxidative degradation of polyamines play a major role in ROS production in the prostate gland. Thus, SSAT enzyme level is a good indication of the ROS production in prostate tissues. Rabbit polyclonal IgG against SSAT enzyme is available commercially (Santa Cruz Biotechnology Laboratory, Santa Cruz, Calif.). This antibody is used to quantitate SSAT enzyme levels in the paraffin sections. High tissue ROS levels cause oxidation of DNA producing high level of ⁸OH-Guanine (⁸OH-G). The ⁸OH-G levels in the paraffin sections is easily detected by IHC method. Highly specific mouse monoclonal antibody against ⁸OH-G is commercially available (Genox, Baltimore, Md.). The IHC of SSAT and ⁸OH-G is performed in two adjacent sections of the same paraffin block. The fluorescence is detected and quantitated using fluorescence-tagged secondary antibody using the AQUA fluorescence quantitation method. All data is normalized using cellular cytokeratin IHC with non-interfering fluorescence spectra in the same slide to specifically focus on the prostatic epithelium and eliminate any interference from the stromal tissue.

While ⁸OH-G levels in the DNA will give an estimation of the oxidative stress history of the prostate tissue, SSAT levels directly relate to the ROS levels at the time of the surgery and formalin fixing of the tissue in the archival paraffin blocks. As we have shown in our studies that ROS levels in the tumor is generally lower than that in the normal tissue, even though they may have more oxidative DNA damage, these two quantitative IHC data in normal and tumor tissues establishes the importance of the dynamics of ROS.

Levels of ⁸OH-G in the nucleus and the SSAT enzyme mostly in the cytoplasm is quantitated. AQUA quantitation of IHC values at each compartment is cataloged and stored. The data from all paired tissue samples obtained from the Clinic samples are compared with the patient follow up record. Five criteria—age of the patient at the time of surgery, pre-surgery and post-surgery follow up for a maximum of 10 years for serum PSA level, tumor recurrence, morbidity and mortality are tabulated with the pathological evaluation (Gleason score) and AQUA analysis of ROS data. The ratio of ⁸OH-G and SSAT fluorescence show the ROS history and red-ox status of the tumor tissue; and if correlation with patient clinical outcome is determined it can be used as a strong prognostic indicator. For example, a high ratio may indicate more prior DNA damage, but a highly proliferating tumor with low ROS concentrations.

### Statistical Considerations:

SSAT enzyme levels, which are a preliminary indication of ROS producing enzyme levels, and ⁸OH-Guanine levels, which are associated with ROS induced DNA damages are recorded as continuous variables. As appropriate, these are subject to log transform and the results are summarized with mean and standard deviation (SD). The differences between the normal and tumor tissues for ROS producing enzyme levels and ROS induced DNA damages are tested by paired t-test. If normality assumptions are not met by the data after log transformation a Wilcoxon signed-rank test is performed. It is anticipated that tissue samples from 225-250 patients will be available. The following table, which is applicable to both the enzyme levels and DNA damage, gives the detectible effect size, i.e., difference in the mean fluorescence scores (after log transformations) between the two groups (one group of cells from normal tissues and the other group of cells from tumor tissues) divided by the standard deviation in scores assuming a paired t-test at two-sided significance level α=0.05. For example, with 250 observations each from the normal and tumor tissues, the effect size of 0.186 in the difference in the log fluorescence scores between the two tissues can be detected with power 1−β=0.9 according to a paired t-test at a two-sided significance level α=0.05.

n

| 1-β | 200 | 225 | 250 | 275 |
|---|---|---|---|---|
| 0.8 | 0.176 | 0.166 | 0.158 | 0.150 |
| 0.9 | 0.208 | 0.196 | 0.186 | 0.177 |

### Additional Analysis:

Additional study analyzes live tissues obtained from the Urological surgery clinics and follow the dynamics of ROS distribution in the live prostatic normal and cancerous epithelial cells using the BD Pathway Bioimager or equivalent for more in depth looks at the production and distribution of ROS in a more dynamic setting

We are comparing cellular ROS levels and the dynamics of subcellular ROS distribution in freshly resected patient tissues and compare with clinical outcome to establish this method for use for prognosis.

### Study Design:

We can collect up to about 75-100 paired normal and malignant prostate tissue sections from CaP patients undergoing radical prostatectomy at the Urological surgery clinics These tissues include the tissues obtained from patients enrolled in chemoprevention clinical trials. The tissues are washed thoroughly in PBS (prewarmed at 37° C.) to remove blood contamination. The tissues are further segregated into two groups.

One group is sliced in 5 mm thick sections, soaked in HEt dye solution for one hour at 37° C. for 60 min following previously standardized conditions before fixing and paraffin embedding. Five mm slices of the paraffin blocks are deparaffinized and the same slide analyzed for HEt fluorescence using both AQUA and BD Pathway Bioimager or equivalent using Hoechst 33342 dye DNA fluorescence as an internal control and the HE. The HEt fluorescence in these tissues, as determined from the Bioimager is validated by the AQUA analysis. Sections from the paraffin blocks are also processed for IHC staining and AQUA analysis for SSAT and ⁸OH-G estimation. All slides are H&E stained after fluorescence microscopy and Gleason score determined.

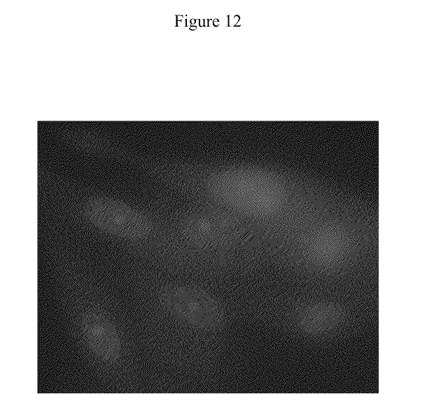

The second group is dispersed into single cells following published procedure and plated on multiple 96-well thin bottom plates. All plates are stained with Hoechst 33342 DNA binding dye (Invitrogen, Carlsbad, Calif.) for nuclear imaging in live cells. Some of the plates are incubated with HEt dye for one hour at 37° C. for dye oxidation and analyzed using BD Pathway Bioimager or equivalent instrument for total ROS level determination. The other plates are equilibrated in the Bioimager incubator and HEt dye are added right before data collection. Kinetics of dye oxidation are determined by fluorescence intensity vs. time plot of subcellular organelles (nucleus, nucleolus, cell membrane, mitochondria and cytoplasm) by segmentation analysis and data integration using BD Pathway Bioimager dynamic data acquisition software and further analyzed for v₀ value calculation. Segmentation of nucleus and nucleolus is straightforward using Hoechst dye (FIG. 12). Mitochondria are segmented using oxidized HEt fluorescence and further confirmed by chloromethyl-X-rosamine (CMX-ROS or MitoTracker Red, Invitrogen, Carlsbad, Calif.) dye. The v₀ of dye oxidation yields the subcellular concentrations of ROS. The dynamics of subcellular ROS distribution is determined by repeating the experiments with plates kept at 37° C. air/CO₂ incubator for 4, 12, 24 and 48 h time intervals.

The total cellular ROS, and their subcellular concentrations and dynamics of production and distribution are important in determining the cellular characteristics. Since AQUA measures only the fluorescence in a fixed tissue, the assay using the BD Pathway Bioimager is unique and appropriate for determining dynamics of not only ROS, but once standardized, other potential biomarker metabolites that can react and change dye fluorescence may also be determined using this method.

### Data Treatment:

The experiments validate the BD Pathway Bioimager quantitation of total cellular ROS with that determined by the AQUA analysis in resected human tissue. BD Pathway Bioimager is then used to determine the dynamics of ROS distribution as a novel and unique method.

### Statistical Considerations:

The cellular and subcellular ROS levels is treated as continuous data. We use Pearson and Spearman correlation coefficients, as appropriate. Optionally, we use polyserial correlation, a method for estimating the correlation between a continuous variable and an ordinal variable whose underlying distribution is continuous. Optionally, we also use survival models such as proportional hazards regression models to test whether ROS enzyme levels and subcellular ROS levels predict time to recurrence or time to death. With a sample size of 75, we will have the following powers to detect different Pearson correlation coefficients, with a two-sided test at a significance level α=0.05.

n

| 1-β | 75 | 100 |
|---|---|---|
| 0.80 | 0.283 | 0.246 |
| 0.85 | 0.304 | 0.264 |
| 0.90 | 0.329 | 0.287 |

### Additional Analysis:

Although we expect patient follow up data within the period, the correlation between subcellular ROS distribution and dynamics may not be enough for statistically significant correlate for use as a prognostic indicator. Validation of the BD Pathway Bioimager method by AQUA, however, will introduce this method for clinical trials and further data collection. In some embodiments, this method is used to follow the efficacy of anti-oxidant drugs that are being used as neo-adjuvants in pre-prostatectomy CaP patients enrolling in chemoprevention clinical trials.

These methods can be used to determine changes in ROS levels and subcellular ROS distributions in freshly resected prostate tumor and normal prostate tissues from patients enrolled in the chemoprevention clinical trials of anti-oxidants as neo-adjuvants.

After validation of the method of determining subcellular localization and translocation of ROS in human tumor and normal prostate cells by AQUA and BD Pathway Bioimager, similar tissues are collected from prostate cancer patients undergoing neo-adjuvant clinical trials. 3,3′-Diindolylmethane (DIM) is being evaluated in this manner in a placebo-controlled, randomized clinical trial that will allow comparative evaluation of ROS. In this study, the effects of DIM are evaluated clinically using a pre-prostatectomy model. Participants diagnosed with prostate cancer and scheduled for radical prostatectomy are randomized to receive 21-28 days of DIM at one of two doses or placebo. The prostate tissue obtained at prostatectomy is evaluated for DIM levels, biologic response to DIM and markers of DIM activity including the androgen receptor, PSA, Ki-67, caspase 3, and DIM-specific markers. This tissue is also available for evaluation of ROS. A difference in the total oxidative stress of the prostate or other epithelial cells obtained from resected tissues can be used as pharmacodynamic data for the neo-adjuvant that allows for determining appropriate dosing regimens. These patients will be followed and any subsequent follow up study and patient outcome is correlated with the tissue ROS levels. In addition to DIM, other anti-oxidants have also been planned for testing future chemoprevention trials that can also take advantage of this method.

Most of the drugs used in Chemoprevention Clinical trials are relatively non-toxic. Thus, it is difficult to ascertain a safe and efficacious Phase II dose within a relatively small Phase I study with 20-30 patients. Since most of these drugs are anti-oxidants, it is contemplated that the drug will reduce the overall oxidative stress in the prostate tissue. Marked reduction of prostate epithelial ROS (close to the stromal ROS) will determine the efficacious (Phase II) dose of the anti-oxidant.

### Results:

Marked reduction of prostate epithelial ROS for patients at a certain dose level can be assigned as Phase II dose. In addition, one may observe a differential change in the subcellular ROS distribution in treated vs. untreated patients. That may suggest a targeted subcellular localization of an anti-oxidant (e.g., mitochondrial localization).

### Statistical Considerations:

The subcellular distribution of ROS is recorded as a continuous variable and, as before, subject to log transform as appropriate and summarized with mean and SD. The change within tissue type (normal or tumor) between the two times in subcellular distribution of ROS is tested by paired t-test on log transformed data as appropriate. If normality assumptions are not met after log transformation a Wilcoxon signed-rank test is performed. The requirement for live cells limits the sample size; and tissue samples from 75-100 patients are available. For both prostate tumor and normal tissues, the following table gives the detectible effect size, i.e., difference between the mean fluorescence scores (after log transformations) at the two times divided by the standard deviation of the difference in scores assuming a paired t-test at two-sided significance level α=0.05. For example, with 75 cells each from the normal (or tumor) tissues, an effect size of 0.290 between the tissues at the two times can be detected with power 1−βP=0.9 according to a paired t-test at a significance level α=0.05.

n

| 1-β | 50 | 75 | 100 | 125 |
|---|---|---|---|---|
| 0.8 | 0.357 | 0.290 | 0.250 | 0.224 |
| 0.9 | 0.420 | 0.341 | 0.295 | 0.263 |

### Methods

Previously published procedures are used for tissue processing, DNA assay and HEt and DCF dye oxidation assays.

Immunofluorescent assay and analysis: Four μm-thick tissue sections are cut and dried at room temperature overnight. All slides are prepared for immunofluorescence following published procedures. Antigen retrieval is done using heat induced epitope retrieval (HIER) with Biocare Medical Bull's Eye for 15 minutes. Endogenous peroxidase is blocked with Biocare Medical Peroxidazed for 5 minutes. The slides are incubated with protein block with Biocare Medical Sniper for 15 minutes to prevent non-specific binding. The primary antibodies (monoclonal mouse anti-cytokeratin, AEI/AE3, Dako, Carpinteria, Calif., 1:200) in Biocare Medical Van Gogh Yellow diluent are applied to the TMA slides and incubated at room temperature for 1 hour. For visualizing epithelial compartment, Alexa 647 goat anti-mouse (Invitrogen, 1:200) in Biocare Medical Van Gogh Yellow diluent is applied to the slide and incubated for 1 hour at room temperature. Finally, the slides are coverslipped with ProLong Gold Antifade Reagent with DAPI mounting medium (Invitrogen) and dried for 30 minutes in an oven at 37° C.

Automated image acquisition: Prostate epithelium is distinguished from stroma with the cytokeratin antibody tagged with Alexa Fluor 647 (red). Membrane/cytoplasmic compartment within the epithelial mask is defined by coalescence of cytokeratin immunostaining. Then an epithelial binary mask is created following pixel-based locale assignment for compartmentalization of expression (PLACE) algorithm, where the stroma is removed. DAPI is used to identify the nuclear compartment within the epithelial mask. The target signal (fluorescent dye) is visualized with the Cy3 filter. Multiple monochromatic, high-resolution (2,048×2,048 pixel, 7.4-μm) 8-bit grayscale images is obtained for each area of interest selected to quantify signal intensity using the 20× objective of an Olympus BX-51 epifluorescence microscope (Olympus, Melville, N.Y.) with an automated microscope stage and digital image acquisition

AQUA Algorithmic image analysis: Two images (one in-focus and one out-of-focus) are taken of the specific tags and the target marker. An algorithm described as rapid exponential subtraction algorithm (USA) is used to subtract the out-of-focus information in a uniform fashion for the entire slide. Subsequently, the PLACE algorithm is used to assign each pixel in the image to a specific subcellular compartment and the signal in each location is calculated. Pixels that cannot accurately be assigned to a compartment are discarded. The data is saved and subsequently expressed as the average signal intensity per unit of compartment area. All the signals in each compartment are then added. The AQUA score is expressed as target signal intensity divided by the compartment pixel area and expressed on a scale of 0 to 33333 (AQUA_{—}1.5, HistoRx). The resultant AQUA score is directly proportional to the number of molecules per unit area. The membrane/cytoplasm compartment defined by coalescence of cytokeratin is used to quantify the intensity of fluorescence of each dye.

Single cell suspension: Epithelial-cell-enriched primary cultures is established from resected prostate. Minced prostate tissue is dissociated with 750 units/ml of collagenase (Sigma, St Louis, Mo.) in F12K tissue culture medium containing 1% fetal bovine serum. This treatment results in the gradual removal of stromal elements from the base of the epithelial cells. After 30 minutes of digestion, aggregates of epithelial cells free of stroma are dislodged from the minced pieces of prostate. These aggregates are washed and plated at high density in F12K plus 10% fetal bovine serum. After 12-16 hours in vitro the unattached cellular aggregates are removed from the culture dishes, washed, and reinoculated into new culture vessels containing fresh medium. After 48 hours in vitro, the aggregates attach to the culture vessels and spread out to yield discrete patches of epithelial cells. By 120 hours in vitro the patches of cells grow and coalesce to form a confluent monolayer of epithelial cells. Ultrastructural examination of these cultures show that adjacent cells are joined by desmosomes and tight junctions and has no filaments and microvilli, giving the cells an epithelial appearance. The cells contain rough endoplasmic reticulum, Golgi apparatus, and secretory granules similar to those of the epithelial cells in the intact organ. In addition, intracellular blebs containing acid phosphatase are observed in the monolayers and are found to increase in size and number with time in vitro. Differentiated function of the cultures are checked by the presence of ornithine decarboxylase and prostatic acid phosphatase by IHC.

BD Pathway Bioimager: Ninety-six well plates are placed in a humidified 95% air/5% CO₂ atmosphere at 37° C. Using the Hoechst dye filter to visualize the nuclei of the cells, nuclear localization and nuclear contour is ascertained and the auto-focus for each well to be standardized. Using the HEt dye filter and the previously androgen treated well (1 nM of 17β-17-Hydroxy-17-methyl-estra-4,9,11-trien-3-one (metribolone) for 45 minutes), the correct exposure time determined the HEt dye for both the nucleus and the cytoplasm at the high ROS level. Pre-warmed HEt dye is added to 11 wells of a row in humidified CO₂/Air atmosphere at 37° C. with a carefully recorded mixing dead-times (usually 30 sec). Fluorescence intensities of Hoechst and HEt in nuclei and cytoplasms for 11 wells in a row are scanned, calculated and stored by the BD Pathway Bioimager running in an auto scanning mode. Four random sections of each well of the 6-well plate take a total scan time of 12 seconds and the is scan repeated every 20 seconds (with a 8 second interval between scans) for 15 minutes. Each kinetic experiment is repeated every 30 minutes for 3 hours and then 24, 48 and 72 h using a fresh well of the 6-well plate.

### Cell Segmentation:

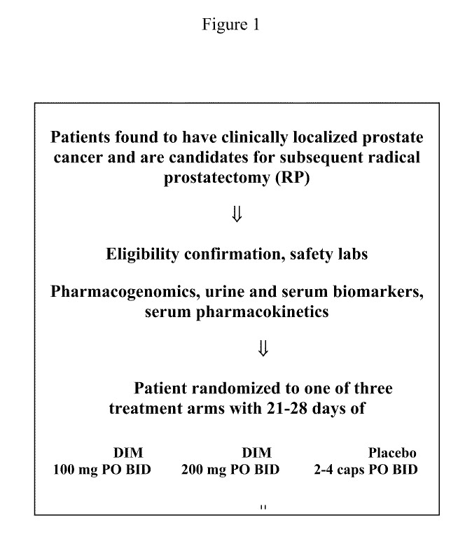

BD Pathway Bioimager software uses fluorescence from both Hoechst and HEt dyes to define the nucleus and the cytoplasm. Parameters of dilation are given to the software to make correct cytoplasmic boundaries. Using the segmentation of the nucleus and the cytoplasm, shown in FIG. 1, the software then calculates the intensity of HEt fluorescence for the cytoplasms and nuclei of each cellular image. In the data re-analysis mode, incompletely segmented images such as contour numbers 11, 12, 13, 22, etc. (FIG. 1) are manually eliminated from the final analysis. Images of about 50 distinctly segmented cells per well will be used for analysis.

### Studies

The quantitative fluorescence data presented here were collected either using a BD Pathway Bioimager (Becton-Dickinson, San Jose, Calif.) instrument (see FIG. 1) or a fluorescence microscope fitted with a CCD camera and AQUA software for fluorescence intensity data collection and calculation.

BD Pathway Bioimager: The BD Bioimager is an automated, confocal, real-time, cell-based kinetic and endpoint fluorescence imaging system containing an inverted microscope and computer aided digital image capture and analysis that has been integrated into a single, compact unit. The Bioimager was designed to provide high-resolution, automated confocal imaging with sophisticated imaging software including cellular fluorescence intensity measurement and cell segmentation and analysis software to assist in developing sophisticated cell- and tissue-based assays at high resolution. All cell-based Bioimager fluorescence quantitation data presented here were performed in 96-well cell culture plates kept in a humidified 5% CO₂/Air incubator fitted with an automatic liquid handler. The Bioimager can read cells in 6-well tissue culture plates and microscopic slides as well. The tissue-based fluorescence in tissue section (s) was assayed in deparaffinized sections on microscopic slides or monolayer cells in 96-well plates.

### (A) Quantitation of Fluorescence Intensities in Cultured Human Tumor Cells Using the BD Pathway Bioimager Using DCFH Dye Oxidation Method:

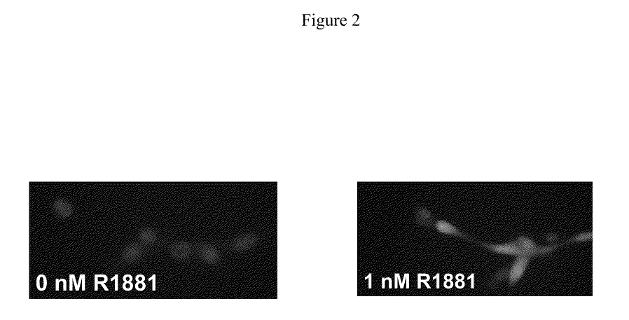

Standardization of cell-based fluorescence quantitation assay. We standardized the BD Pathway Bioimager quantitation of cellular ROS using a well-established cell culture based system. It has been reported that LNCaP human prostate cancer cells produce high levels of ROS after 96 h treatment with 1 nM metribolone (an androgen analog). The ROS levels in these published studies were determined by monitoring DCFH dye oxidation, where DCFH is oxidized to DCF by ROS to yield quantifiable green fluorescence. The representative fluorescence images of the live LNCaP cells captured using the Bioimager are shown in FIG. 2. The DCF fluorescence in live cell cytoplasm and nuclei (FIG. 2B) were quantitated by using the Hoechst 33342 dye staining of the cell nuclei (blue fluorescence in FIG. 2) that allows for auto focusing, positioning and mapping the contour of the cell nuclei and cytoplasms.

### Segmentation and Quantitation of Intracellular Fluorescence Intensities

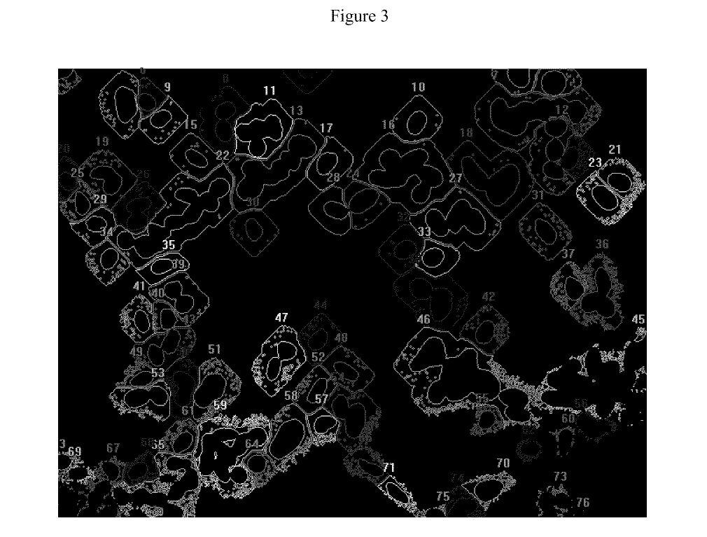

The Bioimager software is enabled to determine the DCF fluorescence in whole cell and cell nuclei using imaging software (Becton-Dickinson, San Jose, Calif.) that maps the nuclei based on the Hoechst 33342 dye fluorescence. The cell contours were mapped based on the DCF dye fluorescence. A representative nuclear and cytoplasmic contour map of LNCaP cells treated with 1 nM metribolone (see FIG. 2) is shown in FIG. 3, where the inner contours represent nuclei. It is noted that cells growing in close clusters (e.g., #11, #13, #16, etc. in FIG. 3) may not always be segmented properly by the software. These cell clusters are manually omitted from overall fluorescence intensity calculation and only the single cells are used for data analysis.

### Kinetic Analysis:

Two types of kinetic studies were performed to determine two different parameter:

(1) Kinetics of DCF dye oxidation: Oxidation of DCFH to fluorescence DCF dye is a relatively fast reaction that is usually complete within 30-60 minutes. Due to the large excess of the DCFH compared to the ROS concentration at any given time point, this reaction follows a pseudo-first order reaction. Curve fitting of the first 20-30 min of the plot of DCF fluorescence intensity vs. time with a pseudo-first order reaction kinetics yields the initial velocity (v₀) values of the reaction (see below), which is directly proportional to the ROS concentration at the time point of DCF dye addition.

(2) Kinetics of ROS distribution at different subcellular domains: Distribution of ROS at different subcellular domains is a relatively slow reaction that may take 24-96 h. This kinetics determines v₀ values of DCF oxidation determined at different time points after incubating tissue sections in PBS for 24-72 h at 37° C. in a sterile CO₂/air atmosphere.

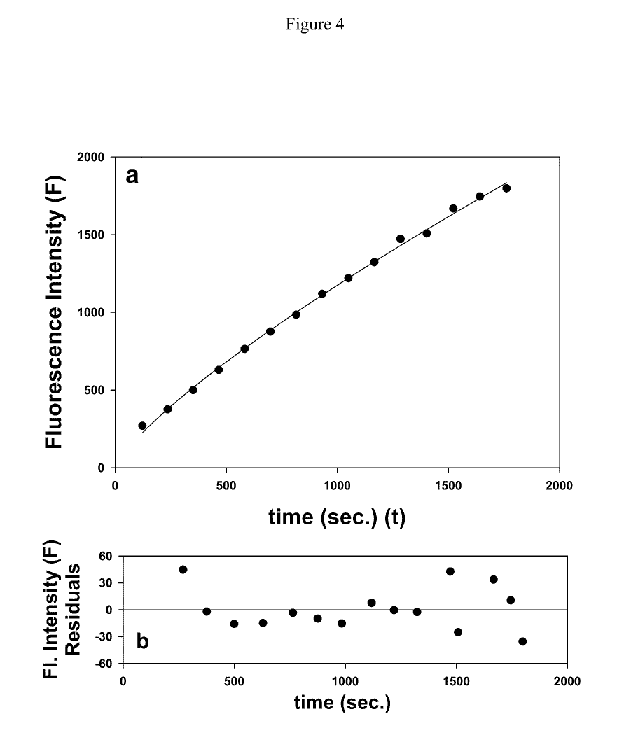

(1) Real-time kinetics of DCF dye oxidation in cell nuclei and cytoplasm: DCF dye oxidation kinetics in the nuclei and cytoplasm of LNCaP cells were determined using the BD Bioimager software coupled with Sigmaplot curve-fitting program. A representative plot of nuclear oxidation of DCF dye in LNCaP cells treated with 1 nM metribolone for 96 h is shown in FIG. 4. The fluorescence intensity (F) at time (t) was fitted to the equation using a Marquardt-Levenberg curve fitting software:

F=A.(1−exp(−B.t)

Where A and B are constants whose values were determined from the curve fit. The fitted curve is also shown in FIG. 4 along with the residuals of the fit. The goodness of fit with randomly distributed residuals of small magnitude (<0.03-2.5% of the original signal) confirm the assumption of a pseudo-first order reaction kinetics. The initial velocity of the reaction v₀:

v₀=(dF/dt)_{hd t 0}=A×B

The v₀ of the DCF dye oxidation reaction:

DCFH+OH. [symbol] DCF+HOH

can be represented by the second order kinetic equation:

v₀=k₂[DCFH].[OH.]

As DCFH dye is in large excess as compared to the intracellular OH. radical concentration at any point of time, the reaction kinetics can be represented by a pseudo first order kinetic equation:

v₀=k₁.[OH.]

Thus, v₀ is directly proportional to the ROS concentrations and can be used as a measure for cellular oxidative stress. This method can be used to analyze any dye oxidation kinetics. Data in each microscopic field takes less than 1 sec to acquire and it takes about 30 minutes to acquire data of a full DCFH dye oxidation kinetics from 12 wells of 96 well plate. This is the first measure of the quantitation of subcellular fluorescence and the real-time kinetics of intracellular dye oxidation.

(2) Kinetics of ROS distribution in subcellular domains:

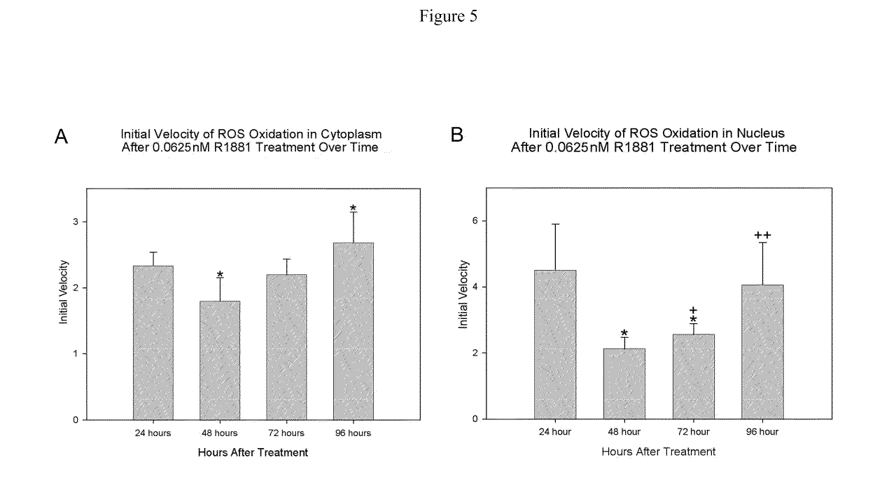

The kinetics of DCFH dye oxidation has been successfully employed to determine the effect of metribolone treatment on the nuclear and cytoplasmic ROS concentrations (in terms of v₀) in LNCaP cells over several time points over 24-96 h time period as shown in FIG. 5. In LNCaP cells, the nuclear and cytoplasmic ROS levels increase over time of metribolone treatment and nuclear fluorescence is always markedly higher than that of cytoplasmic fluorescence (note the difference in y-axes). Thus, this method has now been standardized for the quantitative assay of fluorescence intensity, subcellular concentrations of fluorescence dye and the real-time kinetics of changes in fluorescence intensities or concentrations of the fluorescence dye and/or fluorescence tagged molecules in a cell based system.

Validation of the Bioimager Quantitation Using Immunohistochemical (IHC) Staining of Androgen Receptor (AR) in LNCaP Cells Treated with Metribolone

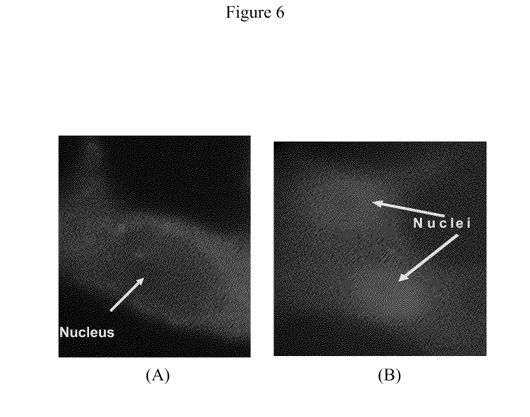

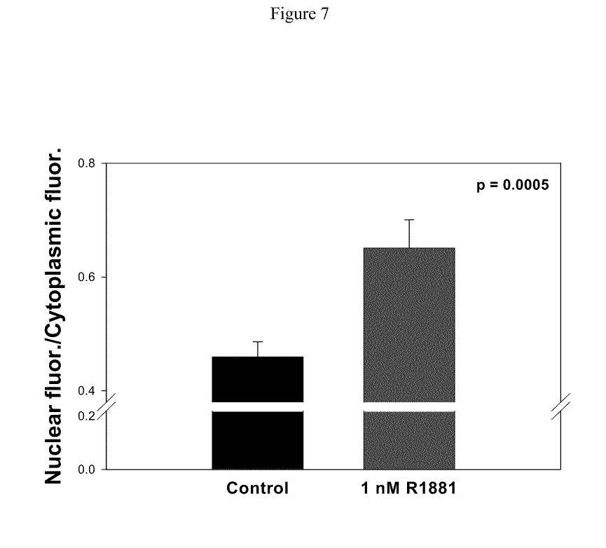

The quantitation of subcellular fluorescence was further validated using a known system—nuclear translocation of AR in LNCaP cells treated with androgen (dihydrotestosterone) or androgen analog metribolone. Even though there are many reports that use IHC, PSA activation and/or western blots of nuclear and cytoplasmic fractions to confirm the nuclear translocation of AR upon androgen treatment of LNCaP cells we are not aware of a quantitative estimation of the levels of nuclear translocation of AR in situ in cells. LNCaP cells in charcoal stripped androgen depleted medium are grown for 6 days in the presence of low (0.05 nM) metribolone and 4 more days in the presence of high (1 nM) metribolone. An IHC picture of cells grown in low and high androgen concentrations showing cytoplasmic vs. nuclear localization of AR is shown in FIG. 6. However, heterogeneity of response was observed in the cell population and only 20 percent of the cells responded as dramatically as shown in FIG. 6. For objective analysis of the response, a quantitation of the IHC fluorescence is necessary. The Bioimager was used to quantitate the IHC fluorescence of AR in the nuclei and in the cytoplasm of about 40 cells from four random areas of the microscopic field from each plate. The average and standard deviation of the ratios of fluorescence intensities in the nuclei and that in the cytoplasm in cells treated with low (0.05 nM, control) and high concentration (1 nM) of androgen analog metribolone are shown in FIG. 7. A 1.5-fold increase in the ratio of nuclear fluorescence intensities over the cytoplasmic intensities was observed in cells exposed to 1 nM metribolone as compared to the cells maintained in 0.05 nM metribolone. The preliminary fluorescence score from 40 control and 40 treated samples (cells) using the BD Pathway Bioimager had means of 5.80 (geometric mean of 329) and 6.41 (geometric mean of 605) with standard deviation of 0.794 and 0.879, respectively, after logarithmic transformation. The observed difference in the mean fluorescence scores between the two groups was highly statistically significant with P<0.002 according to Student's t-test. Thus, BD Bioimager validated as well as quantitated the previously reported observation of the androgen induced nuclear translocation of AR in LNCaP cells.

### (B) Imaging of ROS in Animal and Human Prostatic Tissues Using Hydroethidine (HEt) Dye Oxidation Method

While DCF dye is oxidized by OH. radical, HEt dye is oxidized by superoxide. Because of the better stability and permeability of HEt dye and to avoid the artifact of DCF oxidation by cellular peroxidases, we chose HEt dye to determine ROS level in vivo in animals and ex vivo in freshly resected human prostate tissues.

Changes in ROS in the Prostatic Lumens of Transgenic Adenocarcinoma of Prostate (TRAMP) Mouse Treated with Anti-Oxidant Green Tea Polyphenols

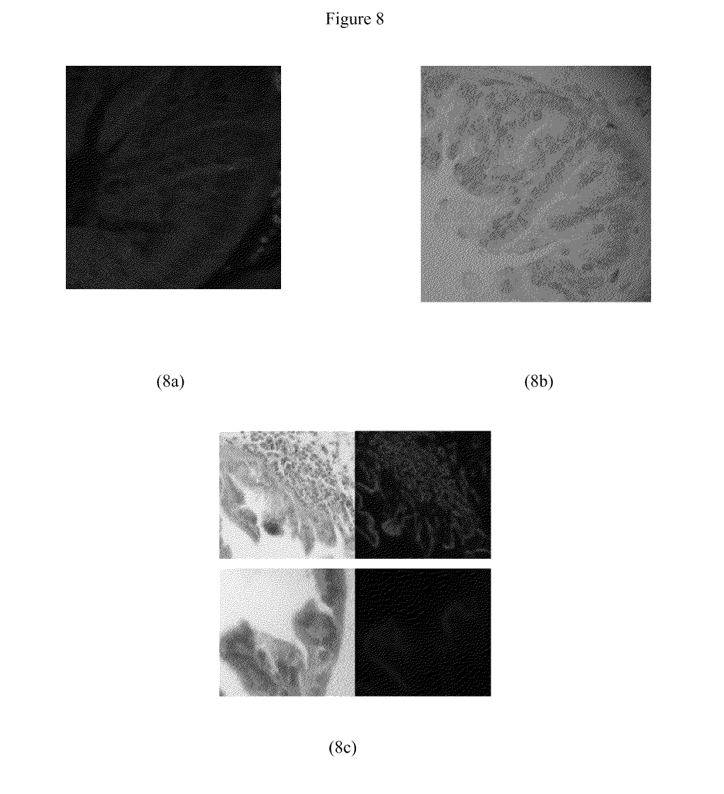

In parallel to standardizing and validating quantitation of cellular fluorescence intensities with the Bioimager system, we also standardized conditions for fluorescence dye oxidation in animal tissues in vivo and in human tissue ex vivo using standard fluorescence microscopy. For these studies, another dye hydroethidine (HEt) was used. HEt in reduced form is colorless. It is oxidized by the ROS superoxide to ethidium (E⁺). The intense fluorescence of E⁺ion can be monitored at 488 excitation/595 emission frequencies at sub-nanomolar concentrations. HEt can be safely injected i.v. into animals one hour before sacrifice. After sacrifice, animals were exsanguinated to reduce fluorescence interference from blood hemoglobin. The tissue was fixed, paraffin blocked and microtome sectioned following a routine procedure. The fluorescence microscopic picture of TRAMP animal prostatic lumen is shown in FIG. 8a. Unlike what observed in DCF dye oxidation assay in LNCaP cells where nuclei are more fluorescent than are the cytoplasms (FIG. 5B), the HEt dye fluorescence in the cytoplasm of the normal prostatic lumen is more than that in the nuclei. Thus, mouse prostate epithelial cells have a different subcellular distribution of ROS than have human prostate cancer cells. This difference of ROS localization may be due to species difference or may be due to a difference in normal and cancerous epithelial cells, or both. This is ascertained when the fluorescence intensities of HEt dye in the human tumor and normal tissue ROS are quantitated, as described herein.

### Diagnostic Monitoring of Prostate Tissue Oxidase Stress Inhibition by CPC-200 Anti-Oxidase Enzyme Inhibitor Drug Administration In Vivo.

Based on compelling evidence, it is now generally accepted that oxidative stress in prostate tissue is one of the major contributors to CaP occurrence and progression (for details see below). It has also been firmly established that androgen is one of the natural agents that induces high oxidative stress in normal and malignant prostate tissues and plays a major role in CaP progression. We have identified androgen-induced activation of the polyamine oxidation pathway as a major endogenous catabolic source of the oxidative stress (see below). Therefore, we focused on developing companion diagnostics for drug agents that inhibit polyamine oxidation directly at its endogenous source, for example to reduce oxidative stress specifically in prostate tissue and thereby, inhibit CaP occurrence, recurrence and progression in high-risk patients with little or no toxic side effects. Our data show that the small molecule polyamine oxidase inhibitor N,N′-bis(butadienyl)-1,4-butanediamine (CPC-200, previously called MDL) § significantly reduces oxidative stress in cultured, androgen-dependent LNCaP human prostate tumor cell line as well as in the prostate tissue in the TRansgenic Adenocarcinoma of Mouse Prostate (TRAMP) model in vivo. CPC-200 treatment also markedly delays the time to spontaneous prostate tumor development in TRAMP model and thereby, greatly increases their overall survival.

To the best of our knowledge, CPC-200 is the first enzyme inhibitor that acts as a prostate-targeted anti-inflammatory oxidative stress modulator drug by blocking a specific metabolic pathway, which is identified as a major source of androgen induced oxidative stress. CPC-200 or its best analog can be initially introduced into the oncology clinics as adjuvant chemotherapy to prevent recurrence and progression of CaP in patients who have been previously treated for primary prostate tumor, but at a high-risk of recurrence (e.g., rising serum PSA). Once the drug is established and approved as a safe agent that can effectively reduce CaP recurrence and progression, it can be further developed as a chemopreventive agent to inhibit carcinogenesis and thus, reduce CaP occurrence in all high-risk men along with the companion diagnostic invention described herein.

It has been well established that the reactive oxygen species (ROS) such as hydrogen peroxide, superoxide, hydroxyl radical, etc. are produced in the prostate gland at markedly higher levels compared to what is produced in most other organs (1). ROS affect prostate tissue by causing inflammation, peroxide laden tumor and suppressor T-cell micro environments, altering cell functions and growth, oxidizing DNA bases and causing mutagenesis, inducing apoptosis and modifying gene expression. The relatively higher levels of ROS and the subsequent chain of events are believed to initiate prostate carcinogenesis, as well as CaP progression. ROS damage can oxidize and mutate DNA and alter the activity of thiol-dependent enzymes. In addition, relatively lower levels of ROS act as cell mitogens and the redox alterations play a key role in a specific signal transduction pathway. ROS are produced during lipid peroxidation and other metabolic oxidative activities. Thus, high fat diets produce ROS, which is proposed to be a cause of the relatively higher incidence of CaP in the industrialized nations as compared to that in developing countries and these men can be at high risk.

Considerable experimental and clinical evidence directly linked increased oxidative stress with an increase in prostate carcinogenesis. Oberley et al used immunohistochemistry to measure oxidative stress-induced damage to protein and DNA in archival paraffin blocks of resected malignant and normal human prostate tissues. Malignant and metastatic human prostate tumor tissues showed convincing evidence of a markedly higher ROS-induced protein and DNA modifications than did the normal prostate tissue. Ho and her coworkers conclusively demonstrated that oxidative damage in DNA and protein is significantly higher in the pre-neoplastic lesions in the TRAMP prostate, as compared to that in the adjacent normal prostate tissues. More recently, published compelling data show that intracellular hydrogen peroxide plays a central role in androgen independent growth and metastasis of androgen dependent human prostate cancer cells. Thus, it is now evident that ROS play a critical role both in the occurrence as well as in the progression of CaP to its more virulent androgen independent metastatic stage. Androgen has been identified as one of the natural agents that induces oxidative stress and carcinogenesis in prostate tissue. Using a well-controlled hydroethidine dye fluorescence assay that monitors dye oxidation by ROS, we directly observed heightened oxidative stress in LNCaP human prostate tumor xenografts in male pude mice in vivo. This increased level of oxidative stress in the androgen dependent human tumor xenografts is significantly reduced within 72 hours after the mice are androgen-ablated by surgical castration, which also causes tumor regression. These data strongly support the hypothesis that androgen induces ROS production in, the prostatic epithelial cells both in culture as well as in vivo.

Thus far, the exact molecular mechanism(s) involved in the androgen-induced production of ROS in the prostate tissue remained largely unknown. We demonstrated for the first time that androgen strongly induces the polyamine catabolic pathway and produces oxidative stress in high polyamine-containing prostate tissues. The prostate glandis unique in having inordinately high (several milli-molar levels) concentrations of the polyamines, spermine, spermidine and putrescine. Other mechanisms that may also lead to an increase in ROS production in CaP cells include expression of nuclear transcription factors including hypoxia-induced transcription factor (HIF-1α), NF-κB, AP-1, etc., enhanced mitochondrial activity and suppression of glutathione S-transferase-π expression with a concomitant reduction in the total cellular glutathione level.

The polyamines, spermidine and spermine, and their precursors, diamine and putrescine, are organic polycations that are present in all mammalian cells. These polyamines are essential for cell growth and proliferation. Since the discovery of spermine in human seminal fluid by Leeuwenhoek in the mid 17^{th} century, the secretion of polyamines by the human prostate gland has been very well studied and documented. The prostate gland is a uniquely rich factory of polyamine production. The semen of healthy men contains a large concentration of spermine (˜3 mM) that originates mainly from prostatic secretion. No other human organ has such high polyamine concentrations and polyamine metabolic activity.

Polyamine catabolism and ROS production are linked. Polyamine catabolism is driven by spermidine/spermine N-acetyltransferase (SSAT), which produces N-acetyl polyamines. N-acetyl polyamines are oxidized by the constitutive enzyme acetyl polyamine oxidase (APAO) generating ROS H₂O₂. DNA microarray and qRT-PCR data clearly demonstrate that androgen induces more than an order of magnitude upregulation of SSAT (the rate limiting enzyme in the polyamine oxidation pathway) mRNA level in androgen dependent LNCaP cells only when exposed to androgen level that causes oxidative stress. Because of the unusually high polyamine levels in prostate cells, high induction of SSAT should induce a large increase in ROS levels and, therefore, should be a major cause of the androgen-induced ROS production in CaP.

We demonstrated that a small molecule, polyamine oxidase inhibitor, CPC-200, almost completely blocked androgen-induced oxidative stress in cultured, androgen-dependent human prostate cancer cell lines and developed a companion diagnostic for oxidative stress. CPC-200 treatment and the diagnostic assay showed an unambiguous reduction in oxidative stress in vivo in spontaneously developing pre-neoplastic lesions in TRAMP animals, greatly delayed the time to prostate tumor development and markedly increased overall survival of TRAMP mice.

CPC-200 is the first small molecule enzyme inhibitor that acts as a prostate-targeted anti-inflammatory oxidative stress modulator by blocking a specific metabolic (catabolic) pathway, which is identified as a major source of androgen induced oxidative stress and inflammation. We standardized a diagnostic method to monitor and develop CPC-200 as a clinically useful prostate cancer chemotherapeutic and/or chemopreventive agent with a companion diagnostic product. We designed, synthesized and screened CPC-200 analogs to identify the most active orally bioavailable analog that reduces oxidative stress in prostate tissue. We obtained a drug and companion diagnostic for the oncology clinic as an adjuvant chemotherapy and diagnostic assay that increases Progression Free Survival (PFS) for the treatment of patients who have undergone surgery or radiotherapy debulking of their primary tumors and have returned to the clinics with rising PSA and Oxidase Stress, but often negative radiographic scans showing no radiographic evidence of a metastatic tumor in the patient. Subsequently, CPC-200 was further tested as a prostate cancer chemopreventive agent for all high-risk men along with the companion diagnostic. We propose to standardized clinical pharmacokinetic (PK) and pharmacodynamic (PD) studies with CPC-200, including a slow release oral formulation for convenient administration.

The diagnostic assays we developed enabled lead drug optimization and identification of the best agent among this class of drug compounds. Synthesis of GMP grade material of the selected agent, stability and large animal toxicity data was necessary for the IND enabling data for the oncology clinic.

Our data clearly demonstrate that enhanced polyamine oxidation is a major contributor to androgen induced oxidative stress and carcinogenesis as monitored in the prostate gland, which is the only human organ that normally contains and also secretes such very high levels of polyamines (˜3-5 mM). We also showed that CPC-200, a specific suicide inhibitor of polyamine oxidase, almost completely blocked androgen-induced oxidative stress in polyamine rich prostate tissue using the invention described herein. CPC-200 treatment was monitored for preventing oxidative stress in chronic inflammation and tumor development in vivo and without any observable adverse side effects.

SSAT induction is a mechanism of ROS production in CaP cells. In our search for the biochemical mechanism and convenient diagnostic monitoring of androgen-induced ROS production in prostate cells, a DNA microarray analysis of gene expression was also performed using the Affymetrix Gene chip Array We used untreated androgen dependent LNCaP cells and cells treated with 1 nM androgen 81881 (a well-accepted androgen analog routinely used due to its stability) for 96 h for these studies. The SSAT (spermidine/spermine acetyl transferase) gene was reproducibly detected in repeat experiments as a highly overexpressed gene in R1881 treated LNCaP cells as compared to untreated control cells. We noted with considerable interest that among the list of genes that are overexpressed by more than an order of magnitude after androgen-treatment, SSAT is the only enzyme that is directly related to a ROS-generating biochemical pathway.

We, therefore, focused on this gene and validated the DNA microarray data by quantitative RNA detection using qRT-PCR. Each data point was an average of readings from six identically treated LNCaP prostate cancer cell sample wells with readings repeated twice in triplicate sets. The data show more than a 30-fold increase in SSAT mRNA level in R1881 treated LNCaP cells as compared to that in the untreated control cells. It was also noted that SSAT is over-expressed only in cells treated with 1 nM R1881, which induces oxidative stress and not in cells treated with 0.05 nM R1881 that does not induce oxidative stress. Thus, 1nM androgen induced SSAT gene overexpression appears to be directly related to endogenous cellular ROS production in androgen-dependent prostate cancer cells and can be monitored by dye oxidation.

We observed that Androgen induces SSAT and CPC-200 drug treatment inhibits APAO enzyme activity. HPLC methods for the quantitation of CPC-200, natural polyamines and their acetyl derivatives in cell extracts as well as in human and animal tissues have been published. This method is routinely used in the analytical laboratory of the core facility of Cancer Centers and was accessible for quantitative assays necessary for the PK/PD determinations. The effect of CPC-200 pretreatment on polyamine and acetyl polyamine levels in LNCaP cells, either with or without R1881 treatment, was determined using this procedure. Each data point was an average of two determinations where sample cell pellets were collected from three independent experiments. R1881 treatment of LNCaP cells at a final concentration of 1 nM for 96 h markedly decreased spermine level, increased putrescine, spermidine, N-acetyl spermidine and N-acetyl spermine levels. These results demonstrated that R1881 treatment, not only increased the SSAT mRNA level, but also enhanced SSAT enzyme activity that catabolizes spermidine and spermine to acetylated products, which in turn are oxidized by Acetyl Polyamine Oxidase (APAO) enzyme to lower polyamine levels. Except for a small increase in the acetyl spermine level, CPC-200 treatment alone has little or no significant effect on most cellular polyamine levels. APAO inhibition following 24 h CPC-200 pretreatment of R1881-treated cells, however, almost completely blocked the R1881 induced increase in putrescine and spermidine by arresting polyamine degradation, which was evident from accumulation of N-acetyl-spermidine and N-acetyl-spermine. These data in addition to a small, but significant increase in N-Ac-spermine levels and a decrease in spermidine levels in CPC-200-treated cells confirmed that CPC-200 treatment at 25 μM efficiently blocked APAO activity as previously reported in the literature. The large increase in the acetyl polyamine levels in the androgen treated cells also indicated that CPC-200 has little effect on the androgen-induced SSAT gene expression and/or SSAT enzymatic activity.

SSAT induction is a major source of androgen induced oxidative stress. In order to confirm that SSAT was the major source of androgen induced oxidative stress, we developed and used a stable LNCaP cell clone transfected with plasmid transcribing siRNA designed to silence SSAT mRNA expression. The SSAT mRNA level in these cells was determined by qRT-PCR method. In the cells transfected only with the vector, the SSAT mRNA level increased by over 8-fold after 96 h treatment with R1881, whereas in cells stably transfected with SSAT siRNA (si22), R1881 under similar treatment condition induced less than 2-fold increase in the SSAT transcript, showing over 75% silencing of SSAT mRNA expression. The ROS production induced by androgen analog R1881 in these cells was measured by DCF dye oxidation assay. The green fluorescence of oxidized DCF in androgen treated LNCaP cells was shown. The effect of SSAT silencing on the R1881 induced ROS production measured by DCF assay was also shown. The ratio of DCF fluorescence (green):DNA fluorescence (blue) for each of the treated well of a 96 well plate normalized to that of the average of control untreated wells represents the ROS level per cell relative to that in control untreated cells. The data points are the mean of numbers obtained from repeat experiments using thirty identically treated wells. R1881-induced increase in ROS production in cells containing the vector alone was comparable to that in untransfected LNCaP cells. In contrast, R1881 induced only a minor increase in ROS levels in cells (si22) transfected with vector transcribing siRNA against SSAT. These data established that SSAT induction is a major reason for androgen-induced ROS production in prostate cancer cells.

### CPC-200 Drug Treatment Inhibits APAO and Blocks Androgen-Induced ROS Production in Human Prostate Cancer Cells.

The ROS levels in LNCaP cells induced by increasing concentrations of R1881 treatment for 96 h, with or without 24 h pretreatment with 25 μM CPC-200, were measured. Each data point was an average of readings from six wells repeated twice in triplicates. CPC-200 treatment alone caused a minor decrease in cellular ROS levels, most probably due to the blocking of the baseline metabolism of polyamine oxidation. The ROS levels of all CPC-200 pretreated cells were normalized to the ROS levels of cells treated with CPC-200 alone. CPC-200 pretreatment effectively blocked R1881 (≧1 nM) induced ROS production. These data confirmed that inhibition of acetyl polyamine oxidase activity by CPC-200 can markedly reduce cellular ROS levels and inflammation in androgen-dependent prostate cancer cells. CPC-200 per se has no known anti-androgenic activity and our western blot analysis has shown no effect

CPC-200 drug treatment inhibited oxidative stress in prostate tissue in TRAMPxFVB animals in vivo, as monitored by in vivo dye oxidation and histology, either with or without immunohistological or immunoflourescent detection of oxidized DNA with antibodies specifically directed to OH⁸dG as a marker of DNA oxidation. We determined the effects of CPC-200 in blocking acetyl polyamine oxidase enzyme catalysed oxidative stress and inflammation in mouse prostatic tissue in vivo. We standardized the in vivo method of Hydroethidine (HEt) dye oxidation. Hydroethidine (8 mg/kg) was intravenously injected into 20-week old TRAMPxFVB animals used in the CPC-200 drug efficacy studies one hour before sacrifice and the prostate gland was removed and paraffin embedded. Prostate tissues in paraffin blocks were sectioned on a microtome, thin sections were de-paraffinized and observed under a fluorescence microscope at 480 nm excitation and 600 nm emission wavelengths. The same slides were then stained with Hematoxylin and Eosin (H&E) for microscopic visualization and digital image analysis. Representative light and fluorescence micrographs of prostate tissues are shown in FIG. 8c. The high fluorescent signals from the HEt reporter dye monitored oxidation was generally located in the prostatic lumens and particularly at the tissue edges with invading inflammatory or pre-neoplastic cells. In contrast, no significant HEt reporter dye monitored oxidation is detected in the normal or nearly normal appearing prostatic tissues of TRAMPxFVB mice treated with six successive administrations of 25 mg/kg CPC-200, injected i.p. once every two weeks. These and other similar repeat data represent the reproducible observations from three individual sets of experimental animals. The results provide clear and convincing evidence that CPC-200 drug treatment inhibits oxidative stress in vivo in the prostatic lumen of TRAMP mice which can be unambiguously diagnosed using the methods and kits and compositions of the invention. Thus, the efficacy of the CPC-200 anti-inflammatory and oxidative stress modulating drug can monitor the inhibition of oxidative stress both in cultured CaP cells and in the prostatic lumen of TRAMP mice in vivo and is a validated diagnostic product for a companion therapeutic anti-inflammatory and oxidative stress modulating drug. Acetyl polyamine catabolism by acetyl polyamine oxidation in the cytoplasm of prostate cells is a major contributor of oxidative stress and in CaP cells as well as in prostatic epithelia and thus Oxidative Stress (along with DNA, RNA, protein or lipid oxidation) as well as inflammation can be conveniently monitored in drug or other treated individuals.

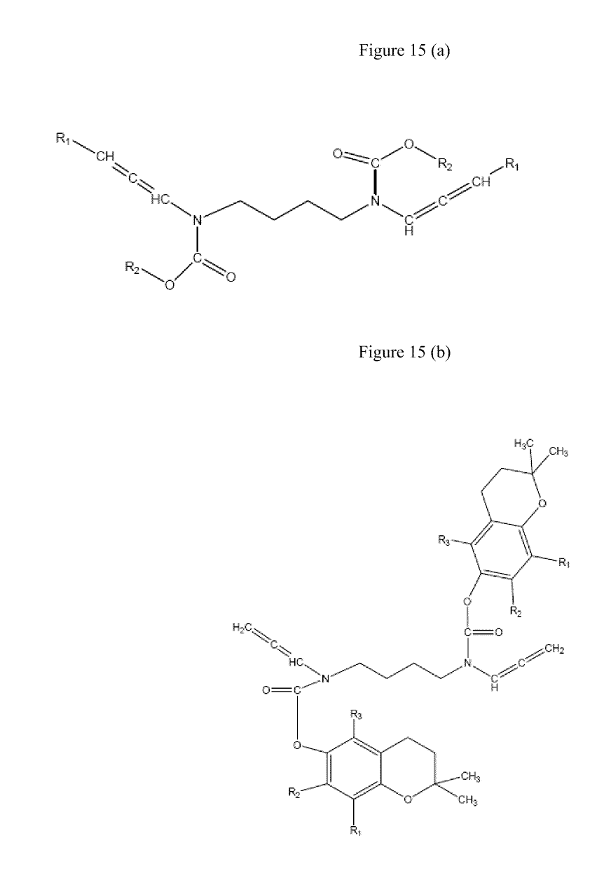

We screened some orally bioavailable analogs of compounds with higher anti-oxidant potential for their ability to block androgen induced oxidative stress in androgen dependent LNCaP human prostate tumor cell line by 2,7-dichlorofluorescene diacetate oxidation assay. The structures of these analogs are shown in FIGS. 15a and 15b. FIG. 15a shows a carbamate analog of CPC-200. It has been previously demonstrated that certain carbamate analogs of biological amines are acid stable and their intestinal absorption is 40 times higher than that of their parent amines. Once at serum pH, the carbamates quickly hydrolyze to release CO₂ and the free parent compound. This is a well used method for developing oral formulation of drugs with low intestinal absorbance. In addition, we have characterized ab analogs of CPC-200 where CPC-200 is connected to a 2,2,5,7,8 pentamethyl 6-chromanol, (an anti-oxidant also present in Vitamin E and its analogs) through a carbamate linkage as shown in FIG. 15b. FIG. 15a shows the Carbam ate derivative of CPC-200 for oral administration. R₁ and R₂ are alkyl groups with carbon chain length between 1-5 including isopropyl and isobutyl groups. FIG. 15b shows the Carbamate derivative of CPC-200 for oral administration. R₁ and R₂are alkyl groups with carbon chain length between 1-5 including isopropyl and isobutyl groups.

One embodiment provides the compounds of Formula Ia or Ib (structures given below).

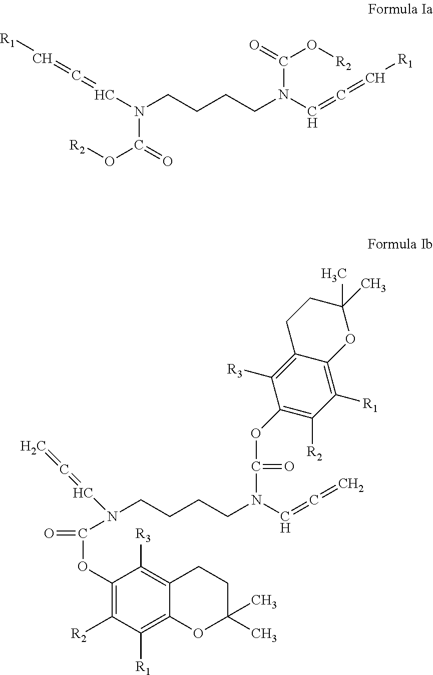

One embodiment provides a method to block androgen induced oxidative stress in androgen dependent LNCaP human prostate tumor cell comprising administration of a composition comprising a compound of Formula Ia or Ib.

Another embodiment provides a method of enhancing the treatment of prostate cancer comprising administration of a composition comprising a compound of Formula Ia or Ib.

Previous studies published from our laboratories have shown that the anti-oxidant PMCoI has considerable cytotoxic, anti-proliferative and also anti-androgenic activities to arrest growth of both androgen-dependent and androgen-independent prostate cancer cells. Because of the carbamate linkage, two PMCoI (or its analog) molecules are released along with one CPC-200 molecule from each molecule of these analogs. The drug analogs shown in FIG. 15b can considerably increase in intestinal uptake of these analog agents, to orally deliver two strong antioxidant (PMCoI) molecules along with one CPC-200 molecule in animals and patients after oral administration to inhibit growth of androgen-dependent cells as well as to prevent progression of the tumor to androgen-independent state. We observed anti-oxidant, anti-androgenic and anti-proliferative properties against LNCaP cells using the DCF oxidation assay.

The ability of CPC-200 and these two analogs (FIGS. 15a and 15b) were tested in doses and formulations inhibiting oxidative stress in prostate gland of TRAMP animals by the hydroethidine dye oxidation methods described herein. Using our standardized an in vivo assay method of determining oxidative stress in animals hydroethidine (8 mg/kg) is injected i.v. into the test mice, and sacrificed after an hour and tissues collected, paraffin blocked, microtome sectioned and observed under light and fluorescence microscopes with digital image analyses. Other sections of the tissue were flash frozen and oxidized hydroethidine and quantitated using a standard HPLC procedure. We also are standardizing an ex vivo hydroethidine dye oxidation method for determining oxidative stress in surgically removed prostate tissue samples following a published protocols. The ex vivo methods provides the necessary technologies to determine PK and PD of the agent. This method enable us clinical validation of surgically removed tissues and peripheral blood lymphocytes from treated and untreated animals or patients for analysis of oxidative stress assays. CPC-200 and the two analogs can prevent prostate tumor formation and/or progression to metastatic tumors in the TRAMP prostate model in vivo and can be monitored using a micro PET-CT scanner.

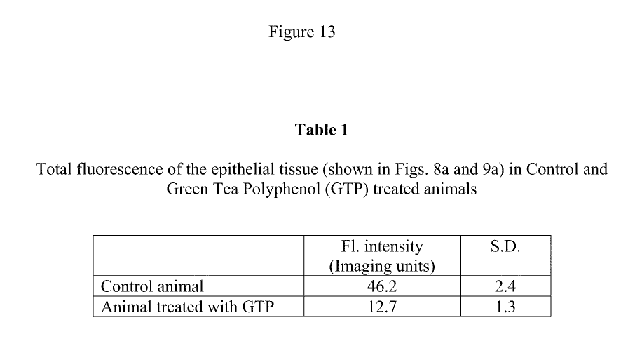

The chemopreventive efficacy of CPC-200 and these analogs against TRAMP tumors is determined as follows: TRAMP females from a homozygous colony are crossed with FVB males to produce TRAMPxFVB (TRxF) male F1 hybrid progeny mice. At 8 weeks of age, prior to development of prostate cancer, TRxF male mice are randomized to vehicle control (n=30) versus CPC-200 (n=30) or analog agent treatment groups. Mice are treated with the optimal dose regimen and A cohort of 10 mice from each treatment will be euthanized at 24 weeks of age, when primary tumors are evident in the control group and the tumors collected for pharmacology, necropsy and other toxicity and tumor burden analyses. Analysis of tissues from CPC-analog and control treated animals confirm the efficacy of CPC-200 and the analogs (FIG. 13) in preventing tumor formation. HEt analysis of the tissue oxidation state performed on a subset of these mice showed the drugs in these formulations inhibited the production of oxidative stress. The remaining 20 mice per condition were followed for survival. Body weights and tumor volumes were measured once weekly throughout the study. A cohort of 10 mice in this treatment group is also monitored over the course of drug treatment by PET-CT scan for tumor growth and metastases. Each mouse is injected with ¹²⁴I labeled NM-404 per standard protocol and imaged on the microPET-microCT scanner. Scans are performed at age 8 (start of drug or analog treatment), 12, 18, and 24 weeks for a longitudinal analysis which is sufficient time gap between scans to allow for the clearance of ¹²⁴I labeled dye and showed PCa tumors in the control animals but no prostate tumor in the drug treated animals.

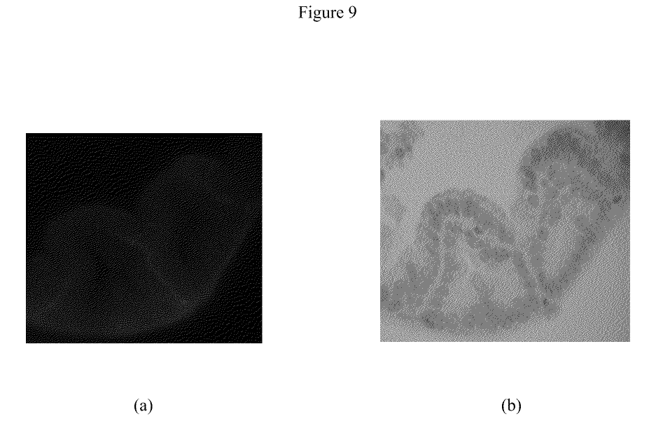

The HEt dye oxidation method was also employed to determine the in vivo efficacy of anti-oxidant Green Tea Polyphenols (GTP) in reducing oxidative stress in TRAMP animals. The animals were fed GTP (0.2% w/v) in drinking water every alternate day for two weeks before injecting HEt dye 1 hour prior to sacrifice. The Ethidium dye fluorescence of the prostatic lumen in GTP treated mice is shown in FIG. 9a. A quantitative estimation of HEt fluorescence FIGS. 8a and 9a are shown in Table 1. The data shows a clear effect of GTP I in reducing oxidative stress. The fluorescence intensities of the prostatic epithelial cells in patients should more objectively ascertain the anti-oxidant activity of the drug. This also helps ascertain the relative ability of different anti-oxidants in reducing the oxidative stress in animal prostate and other tissues.

### ROS in Resected Human Prostate Tumor Section

Surgically removed human prostate tumor tissue sections were obtained. The tissue type was determined from pathological analysis at the surgical pathology and the normal tissue was separated from the tumor tissue following routinely used surgical procedure.

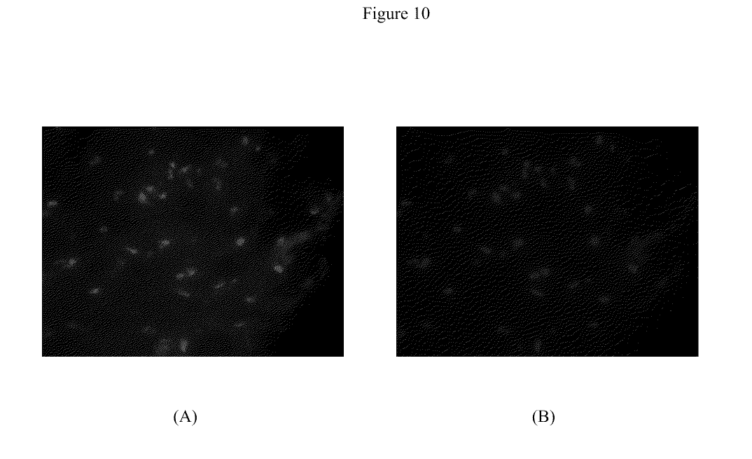

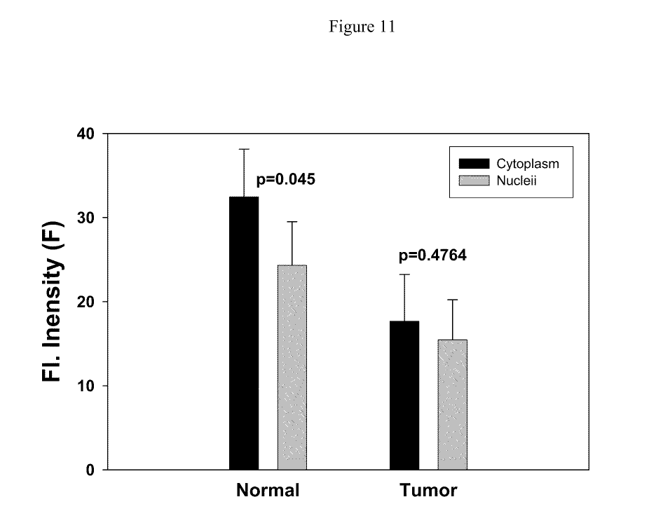

The tissues were cut into 5 mm slices and were incubated in 10 uM HEt dye in isotonic posphate buffered saline at 37° C. in a 5% CO₂/95% Air incubator for 1 hour. The tissues were then formalin-fixed, processed, paraffin blocked and microtome sectioned. On the day of the experiment, the slides were deparaffinized and incubated in Hoechst 33342 dye for 15 minutes. The slides were then washed, dried and observed using BD Pathway Bioimager. The Bioimager pictures of a representative human tumor tissue are shown in FIG. 10. The total fluorescence intensity data of matched normal and tumor tissues from nine patients have been analyzed thus far. These data along with their corresponding Gleason scores have been listed in Table 2. Our data with six cases show consistently that the tumor tissue has less ROS than has the normal tissue. Therefore, even though tumors may have more ROS induced DNA damages, those damages may be prior to its malignant transformation. Once transformed, the actual ROS levels may be less in the tumors. All tumor tissues analyzed to date are from tumors with relatively low Gleason score (<7). More tissues at different tumor grades will be analyzed for confirmation. We analyzed resected prostate tissues and compared the data with patients' clinical outcome. One pair of normal and tumor tissue obtained from the same patient was further analyzed for cytoplasm and nuclear distribution of ROS using the AQUA method. The data are shown in FIG. 11. Both nuclei and cytoplasm of the normal tissue have relatively higher ROS levels than have either the nuclei or the cytoplasm of the tumor tissue (as shown in Table 2). When segmented into nuclei and cytoplasm, there is a significant difference between nuclear and cytoplasmic ROS in normal tissue, but no difference between normal and cytoplasmic ROS in the tumor tissue. Further analysis of the ROS distribution with the BD Bioimager with more tissues is expected to firmly establish this difference.

Live prostate tissue isolated from one patients' tissues exposed to HEt dye and stained with Hoechst 33342 were observed using the BD Bioimager confocal microscopy, where the nuclear, nucleolar, cytoplasmic and mitochondrial fluorescence were monitored in 15 confocal sections. One representative image of a confocal section is shown in FIG. 12. Use of mitochondria specific dye for segmentation analysis of these subcellular organelles and kinetics of dye oxidation and ROS distribution of normal vs. tumor cells from patients' tissue samples and patient follow up data firmly establish tumor characteristics and is expected to yield a prognostic indicator.

In summary, (A) BD Pathway Bioimager was used to standardize quantitative estimation of subcellular ROS levels and ROS translocation in real-time in LNCaP human prostate cancer cells, mouse and human prostate cancer tissues exposed to androgen. In addition, (B) in vivo and ex vivo HEt dye oxidation assays were used to observe oxidative stress in animal and human prostate epithelial cells in situ. Mice treated with anti-oxidant Green Tea Polyphenols showed a marked decrease in the oxidative stress in their prostatic epithelial cells. These ex vivo method of ROS estimation can now be combined with quantitative measurement using the AQUA and the BD Pathway Bioimager to determine subcellular localization and real-time kinetics of ROS production and also quantitate enzymes directly related to ROS production in the prostate in surgically resected animal and human prostate tissues. These data when combined with patient follow up data may yield valuable information about the role of ROS as a prognostic indicator of prostate cancer progression as well as for determining chemopreventive abilities of anti-oxidant drugs undergoing clinical trials. This method, however, can also be standardized for any metabolomic studies of animal and human cells and tissues where the metabolites or metal ions such as Ca²⁺ can be fluorescence tagged and its subcellular distribution can be determined in real-time. Thus, in some embodiments the method described herein satisfies a long awaited need of tracking and quantitatively determining small molecules and metabolites in resected human tissue that in other embodiments is used for diagnostic and/or prognostic purposes.

Advanced hormone refractory metastatic prostate cancer (CaP) is the second leading cause of cancer deaths among US men. In 2007, over 218,000 US men are projected to be diagnosed with CaP and over 27,000 men are anticipated to die of this disease. Aberrant production and distribution of reactive oxygen species (ROS) such as H₂O₂, hydroxyl radical, superoxide, etc. are found to be critically important in carcinogenesis as well as in regulating cancer progression and metastasis. National Cancer Institute (NCI) sponsored CaP chemoprevention clinical trials of anti-oxidant drugs as neo-adjuvants are currently ongoing at several hospitals and institutes nationwide. Due to a lack of a clinically applicable method of determining tissue oxidative stress, there exists no good method of determining the pharmacodynamic parameters of these anti-oxidant or oxidative stress modulator drugs that may help determine drug doses or predict drug effectiveness or patient risk. In order to determine real-time intracellular production, subcellular localization and intracellular trafficking of the small molecules and other metabolites such as ROS that may have profound diagnostic and prognostic implications, we will use a BD Pathway 855 Bioimager and data analysis software to acquire, process and quantitate the fluorescence intensities of cellular and subcellular domains in live cells. The cells are obtained from the resected human tissues and tissue biopsies. Using hydroethidine dye (HEt) that is oxidized by the ROS to highly fluorescent ethidium ion (E⁺), we have standardized a fluorescence microscopic method of observing ROS levels in animal prostate and other tissues in vivo. We have also standardized conditions for ex vivo HEt dye oxidation for estimating ROS levels in the prostate and other tissues resected from animals and humans. Here, in some embodiments are methods to standardize the use of BD Pathway Bioimager instrument to quantitate the cellular ROS and their subcellular distribution and translocation in the resected patient tissues or tissue biopsies. In other embodiments, the data collected is used as a prognostic indicator in patient follow up

We have used the AQUA method to quantitate ROS producing enzyme levels and ROS induced DNA damages in matched resected prostate tumor and normal prostate tissues from prostate cancer patients, who underwent radical prostatectomy. We compared cellular ROS levels and the dynamics of subcellular ROS distribution in freshly resected patient tissues and compare with clinical outcome to establish this method for use for prognosis. We determined changes in ROS levels and subcellular ROS distribution in freshly resected tumor and normal prostate tissues from patients enrolled in the chemoprevention clinical trials of anti-oxidants as neo-adjuvants.

The examples and embodiments described herein are for illustrative purposes only and in some embodiments, various modifications or changes are included within the purview of disclosure and scope of the appended claims.

1. A method for differentiating prostate hyperplasia, inflammation and cancer tissues based upon oxidative stress status comprising:
fixing a tissue sample;
treating the tissue sample with a fluorescence indicator; and
analyzing the treated tissue sample with a fluorescence microscope.

2. (canceled)

3. (canceled)

4. A method of determining the efficacy of anti-oxidant chemopreventive or chemotherapeutic agent for the prevention or therapy of prostate inflammation and cancer comprising:
fixing a tissue sample;
treating the tissue sample with a fluorescence indicator;
analyzing the treated tissue sample with a fluorescence microscope; and
comparing the sample fluorescence to a control sample.

5. The method of claim 1 or 4, wherein the fluorescence indicator is selected from dichlorofluorescein diacetate, or hydroethidine dye.

6. The method of claim 1 or 4, wherein the tissue sample is a resected tissue sample.

7. The method of claim 1 or 4, wherein the analysis with a fluorescence microscope further comprises employment of the automated quantitative analysis system (AQUA).

8. A method for differentiating prostate inflammation and cancer tissues based upon oxidative stress status comprising:
treating a live animal with hydroethidine dye;
obtaining a tissue sample; and
analyzing the treated tissue sample with a fluorescence microscope.

9. (canceled)

10. (canceled)

11. (canceled)

12. (canceled)

13. (canceled)

14. (canceled)

15. (cancelled)

16. A kit for the quantitative ex vivo estimation of reactive oxygen species in a prostate tissue sample comprising a fluorescence indicator responsive to reactive oxygen species and instructions for use of the kit.

17. (canceled)

18. (canceled)

19. (canceled)

20. The kit of claim 16, wherein the fluorescence indicator is hydroethidine dye.

21. The kit of claim 16 or 20 wherein the instructions for analysis of the data further comprise a comparative dataset correlating concentration of SNPs and reactive oxygen species to prostate cancer progression, chronic non-resolving inflammation, immune disfunctions and metastasis.

22. (canceled)

23. (canceled)

24. (canceled)

25. (canceled)

26. (canceled)


SEQUENCE LISTING (18 sequences)

SEQ ID NO: 1 - skipped

SEQ ID NO: 2 - DNA, 839 bp, Homo sapiens
aagccacaggctccctggctggcgtcagctaaagtggctgttgggtgtccgcaggcttctgcctggccgccgccgcctataagctaccaggaggagctttacgacttcccgtcctgcgggaagtggcgggcacgatcgcaaggtagcgcagaagcttctcaatggccagcgccagctgcagccccggcggcgcactcgcctcacctgagcctgggaggaaaattcttccaaggatgatctcccactcagagctgaggaagcttttctactcagcagatgctgtgtgttttgatgttgacagcacggtcatcagtgaagaaggaatcggatgctttcattggatttggaggaaatgtgatcaggcaacaagtcaaggataacgccaaatggtatatcactgattttgtagagctgctgggagaaccggaagaataacatccattgtcatacagctccaaacaacttcagatgaatttttacaagttacacagattgatactgtttgcttacaattgcctattacaacttgctataaaaagttggtacagatgatctgcactgtcaagtaaactacagttaggaatcctcaaagattggtttgtttgtttttaactgtagttccagtattatatgatcactatcgatttcctggagagttttgtaatctgaattctttatgtatattcctagctatatttcatacaaagtgttttaagagtggagagtcaattaaacacctttactcttaggaatatagattcggcagccttcagtgaatattggtttttttccctttggtatgtcaataaaagtttatccatgtgtcagaaaaaaaaaaa

SEQ ID NO: 3-6 - skipped (4 entries)

SEQ ID NO: 7 - protein, 72 aa, Artificial Sequence
  Description of Artificial Sequence: Synthetic polypeptide
MASASCSPGGALASPEPGRKILPRMISHSELRKLFYSADAVCFDVDSTVISEEGIGCFHWIWRKCDQATSQG

SEQ ID NO: 8-12 - skipped (5 entries)

SEQ ID NO: 13 - DNA, 2446 bp, Artificial Sequence
  Description of Artificial Sequence: Synthetic polynucleotide
cagtgcagctgagatcagacttccatgtgtaactcccactaccctaccaggatgccttttcataaaggtaagaaatgtaaatttggccttaatatacaaagttgccagggcagcactgggtcagttctacatatagtacttctacgttcatcagcggaaactttaagggaaggtgaaaatgcttctagaaggcgactggacaccagcgcctttgggctcttcttctaaggccaatagtgacctaaattattgactgactgctccaatcaagtgggcaaaagggtaccaaggccgccaacatcagacaaattcacttgagggcctacctatgtgctttgaaagacaaaactgctgttgtgaaggacactgtatttcagaaaaacataatcatattaacaactagtaacaatgtaaaatgctgatgtgttgaatgctactttagaaaaacatgttaaaatctacaaaaaaaaatttatgatacaaaactacgttatcaatcatctagctagctaactatctacagacatggttttcatttctgttgctcaggatggaaagcagtgggatgatcatagctcactgcagccttgagctcctggcctcaagtgatcctcctgcctcctaagtagctggggccacaggtggacacagtgacacctgggttttttcttttgtagagacagggtttcactacactgcccaggctggtgtcaaactttggagtctcgctgtgtcacccaggctggggtgcagtggtggggatctcggctcactgcaacctctgccacctgggttcaagcgattacttcctgtatcagcctcccgagtagctgggactacaggcatgtgccaccacacccggctaatttttgtattttttgtagagaactgggtttcaccatgttggccaggctggtctcaaactcctgacctcaggtgatccacctgcctcggcctcccaaagtgctgggattacagtcatgagccgatgaacactttcttatgctattaaatggcctaacccaggcgggtcgtggtggctcacgcctgtaatcccgaaaactcggatggccaaggtaagaagatcacttgaacctaggaattccaaactggcctgggcaacatagcgagactcccatccctacaaaagatacaaaaattaggcctggcgcacaccacgctcggctagtttttgtaccttttgtagagacagggttgcgtcatgttgcctaagccggtctcgaactcctgaacccaagccatccatcctcccgcctcggcctcccaaagtgctggagattacaggggcccagccagcctcatgttttccttttaagcagtcccttccctgttgcacacttgggttagttttctttttaatttttttaaaacagggggttacctcaatctcgcagcctggagtgctgtggtgggatcacagctcattggagccttgaaccttggggttcaagtagctggggggctgaggtaggactacagagatggggtcccgccatgttgccaggctgctcttggcctgaagggcatctcccgctggccgtgcccggacatagttttctatttttgaccgacataaacactgtgctgagtcggtgtttgtcaacacacaggacctggcggggaggtcgcggttaccaggctccactctaagtagaagactgcccagctccaagcactgtacctcccggtgacgtcgccgaacgcccgccctgtgacgatacctaaggcccaccttcatgacgccgccgaaggcccgcccctgtgacgccgccggaggcccgcccctcacgcggagccaatcggaactcgaggcggggctgttgggtcttcgggagcgcgcatgcgcggggggccacaggctccctggctggcgtcagctaaagtggctgttgggtgtccgcaggcttctgcctggccgccgccgcctataagctaccaggaggagctttacgacttcccgtcctgcgggaagtggcgggcacgatcgcaaggtagcgcagaagcttctcaatggccagcgccagctgcagccccggcggcgcactcgcctcacctgagcctgggtacgtgcagccccacaacaccttccccagccagggcccggggaccccgggagcgtcccccgccacctggcgccgctcatacctgggcaagggtgggaccccactgaggcccgccacgcattagggagcttgcacttcccgagttttgacctctgacgggcagttgtaatagcattaaagtttttgaaattttgtagcgggggtagaaggggcttggaaagggaagaaaacatcttttaaaatataacgttccggccgggcccgggggttaaccctttaatccacatttgggaggccaggcaggggattacgaggtaggagttaagaccacctggccgcatggggaaccctgttttactaaa

SEQ ID NO: 14 - DNA, 1260 bp, Artificial Sequence
  Description of Artificial Sequence: Synthetic polynucleotide
  modified_base (75)..(75): a, c, t, g, unknown or other
  modified_base (128)..(128): a, c, t, g, unknown or other
  modified_base (147)..(147): a, c, t, g, unknown or other
  modified_base (157)..(157): a, c, t, g, unknown or other
  modified_base (220)..(220): a, c, t, g, unknown or other
  modified_base (232)..(232): a, c, t, g, unknown or other
  modified_base (249)..(249): a, c, t, g, unknown or other
  modified_base (295)..(295): a, c, t, g, unknown or other
  modified_base (1246)..(1246): a, c, t, g, unknown or other
  modified_base (1259)..(1260): a, c, t, g, unknown or other
gagatatagatggggagatttttaatttagttttttatttaaaattgtgtttataaggaaagagattattatgtnttttgatagagaatttgggagagtttgtttaatttagagagagatgggttagnaagatttatagggttggantgtgatttangaaaagatttaaggtgtttggagaataatttttggggaggaatttttggaataaaaagaaaantttggtaaaaanggtggatttggttttanatgaaataaaaaaattaaccggatgtggttgcacacgccctgtatncccagctactcaggaggctgaggcacgagaatcactagaacccaggaggtggaggttgcagtaagccaagttcgtgccactaccctccagcctgggcaacagagtaagactccatctaaaaaaaaaatgaagaagaagaaattagtgtagtgtgggaagtgaaaaaaaaaaaaagaaaaggaaaagaaaaatgattgaattcatgaatacactcttatgtggcctgcaccgactttgacacaaattagattggcttagtaggcaagggtgggatcttttcataattttatttgatgtctaaaatacatttatctttttttcttataggaggaaaattcttccaaggatgatctcccactcagagctgaggaagcttttctactcagcagatgctgtgtgttttgatgttgacagcacggtcatcagtgaagaaggaatcggtgagctagccaaaatctgtggtgtagaggacgcggtgtcagaaatgtagggatagcatttattcactttatgaaatgataaagaattttttttttttttttttttgagacagagtctcactctattgcccagactggagtgcagtggcacaatcttggcttactgcaacctccacctcccaggttcaagcgattatcctgcctcagcctcctgagtagctgggattacaggcgtgtgcaaccacatccggctaatttttttatttttagtagagatggagtttcaccatgttggtcaggctggtctcaaactcctgacctcgtgatccacccacctcagcctcccaaagtgctgggattacaggcgtgagccaccatgcccggccatgaaatgataaagaatttctaaagggtggctgttttggatgaagtgctggaccctggctataaaagatgagcactaggctttttctctcaccccttagagttgaatttgatatattgagaactntgctatcgctccnn

SEQ ID NO: 15 - DNA, 4906 bp, Artificial Sequence
  Description of Artificial Sequence: Synthetic polynucleotide
cacccacctcagcctcccaaagtgctgggattacaggcgtgagccaccatgcccggccatgaaatgataaagaatttctaaagagtggctgttttggatgaagtgctggaccctggctatggaacatgagcactaggctttttctctcaccccttagagttgaatttgatatattcattcatttatataaatatttatggcaaaaaaatatggataagacatgttcttagcttgattaggggaatgatgaccatcactaaccaaagaagatggcctagggggcagggctcaagtgtccagtggctttgtgtcatggttgaaagtgtggccttagtggttccctggaaaggttcagagtctcttggtggatcctgggtcagagcccctctctccctccctccctcctgccctcccacctcctgccctgcagctgggcaccaccctctgcagccccagtcccccagtcatgcaccatgtcattttcttttttttttttttttcaggacggagtctcgctctgtcaccaggctggagtgcaatggtgcaatctcggctcactgcaacctccgcctcctgggttcaagcaattctcctgcctccacctcctgagtagctgaaactacaggcacgcaccaccacacccggcttatttttgtatttttagtagagatggtgtttcaccatgttggccagcctggtcttgaactcctgacctcgtgatctgccggcctcggcctcccaaagtgctgggattacaggcatgagccaccacactcggccaccattttattttcaactccctttcatggaagaacgtttagcctttggtctctttcttgatttatgacagctcgcggctttcaagaaaactacctatgaataggctgtgtaacttttatttatttatttattttttgagatggagtctctgtcttctaggctggggtgcagtggcatgatctcggctcactgcaacctccccctcccaggttccagcaattctcctgcctcagcttcccaagtagctgggattacaggcatgcaccaccacacctggctaatttttgcattttttttttattagagatggggttttgtcatgttggccaggctggtctcgaactcctggcttcagatgagctgcccacctcagcttcccaaagttttggcattataggcatgagtcactgcacccagcttgttaatgttattttcaagcacaccttcaaaagtttattccaaggccttgctctcatagcagcaaagcctgtctgttgcatagtggggctcttgttagcatcttccttttggtggatgttgtgtagacccggggcagtaaggcatgttaacctcgaggacattggacctggctggcctgactgttgggtctccctcctaggacatggcgagccatgggtggggcagtgtctttcaaagctgctctcacggagcacttagccccaatccagccctccagggagcaggtgcagagactcatagcagagcaccccccacacctgacccccagcataagtaagaggagccgctgctccaggtgtatttcagcaccagtgttggggagcatgtcctcctgagagcatctaacgattgctttagagagccctctgggtggtttatttattttcatttttattttttaattttattattttaaaaatatttatttatttatttatttatttatttatttatttatttattgaggcagagtctcgctctgtcgcccagactggagtgcaatggtacgatatcggctcactgcaacttctgcctcccgggttcaagtgattctcctgcctcagcctcctgagtagttgggatttcaggcacccaccatgccaggctagtttttgtatttttagtagagacaggatttcaccatgttgtttaggctggtctcgaactcctgacctcaggtggtctgcccgcctcagactcccaaagtgctgggatcacaggcgtgagccactgcgcctggcaaaattttatttatttattttttttgagacaaagtctcactctgtcgcccaggctggagtgcagtggtgcaatctcaactcactgcaacttttgcctcccaggttcacacgattcttctgcctcagcctcaggagtagctgggattacaggcgcctgtcaccacacctggctaatttttgtatttttagtagagatgggtttcgccatgttggccaggctggtctcgaactcttcatctcaagtgatccacctgcctcggcctcccaacgtgctgagattacaggtgtgagtcaccacacctggcttattatttttttttttagattcagagtctcattctgttgcccaggttggagtacagtggtataatcatggcccactgcagcctcgaactcctggggtcaagtagccctcccaccttagcctccttagtagtagggactgcaggtgaatgccaccgtgcccagctaattaaagaatttttttttttttctgaaagtcttgctttgtcacccaggctggagtgcagtggcatgaacacagctcactgcagcatcaatcttctgggctcaagggatcctcctgcctctgcttcctaagtagctgggactacagacataggccaccatgccaattttttttttttttttttgagatggagtctcgctctgttgcccaggctggagtgcagtggcgccatcttggctcactgcaagctccacctcctgggttcacgccattctcttccctcagcctcccgagtagctgggactacaggcgcctgccaccacacctgactaattttttgtatttttaatagagacggggtttcaccatgttagccaggatggtctcaatctcctgaccttgtgatccacctgcctcagcctctcaaagtgctgggattacaggcgtgagtcaccgcgcctggcccaccatgcctaattttaaaaaaaaaatttttttatagacatagcatctcactacattgcccaggctggtcttgaacttgtgggcttaagtgaccaattttcacaactcagcctcccaaagtgttggaattataggtttaagacactatacccagcctttaagaatcttcttgtagagatggaatcttgctgtgttgcccaggctgttctcaagctccttgcctcaagtgattttctaccttggcctcccaaagtgctggaattaaaggcatggactgtgtggttcttctggcacttacacgtggtcttgtctggccggttgtctggtcctgtctgtttctgcctttcctctttctccagggaaaacctaagctttcctttttgtcctcatcttgtgtttttctgggtccatgggcagagtagagttctagaatggtttcctaaagcagccaagccctaccctttgatttctaagtacatttagaaaaacacatataaggctgggcatggtggctcacgcctgtaatcccagcactttgggaggccgaagtggatggatcaccaggtcaggagttcgagaccaggctaaccaacatgatgaaaccctgtctctactaaaaatacaaaaataagccgggcacggtagctcacgtctgtaatcccagcactttgggaggccaagatgggtgtatcacctgaggtcaggagttcgagaccagcctggccaacatggtgaaacccagtctctactaaaaatacaaaaaaagtagctgggcgtggtgataggcgcctgtaatcccagctacccaggaggctgagggaggaaaatcactggaacccgggaggcagaggttgctgtgagccgagatcatgccagtgcactccagctagggcaacagagcaagattccatctcaaaaaaaaaaaaaaaaaaaattagccgggcattgtggcatgggcctataatactcaggaggctgaggcaggagaagcgcttgaacccgggaggtagaggttgcagtgagccaagattgcagccttgcactccagcctgggtgacaagagtgaaactctgtctcaaaaaataaagagaaaaacatatagaaaacattaacaccccaggcagtataccttgtcaaacatacctcaggcaaatgcattcaggagaagaaaatacatcttatttccctcttcatgtttcgttttttttttttgttttcttttgtattactcagtgttgggtattttgtattttattttgcagggagctggtaagtcgcctacaggagcgaaatgttcaggttttcctaatatctggtggctttaggagtattatagagcatgttgcttcaaagctcaatatcccagcaaccaatgtatttgccagtaggctgaaattctaccttaatggtaagatgttaacggtaacatgttccctttcttagcagttccattattcagtattctgggtaatgtcttttggaatgcaacttgaacagtcacatcagagttaaatattgagatgaatggttctctttttttttttttttttttttttgagacggagtctcgctctgtcgcccaggctggggtgcagtggcgcgatctcggctcactgcaagctccccctcccgggttcacgccattctcctgcctcagcctccccagtagctgggactacaggcgcccgctaccatgtcaggataattttttgtatttttagtagagacggggtttctccgtgttagccaggatggtgtcgatctcctgacctcgtgatccgcccgcctcggcctcacaaagtgctgggattacaggcgtgagccaccgcgcccggccgagatgaatggttctcttaattgatgtcttttgccctttggtaccctttgctcagcaaaacagacttagatcatcacctgtcttagctttattatctataacatcacctgtctgtacataaatgtctgcatttcg

SEQ ID NO: 16 - DNA, 409 bp, Artificial Sequence
  Description of Artificial Sequence: Synthetic polynucleotide
gtgggctcagccatcctcccaactgagcctcctgagtagctgggactacaggtgtgagccatcacactcaactggtgtagctattttagaagacaaactggcagtttctcaaaaggctaaacatacagtcatcatataatgcaacaatttcactcctaggcatatatcccagagaaatagaaatatatgtccacacaaaaacttgtacagcaatcttcatagcagcattgttcataatagccaatacgtggaaacaacccaaatgtccatcaactgatgaacagataaacaaaatgcagtgtgtctctaccatggaatattattcagccacagaagaaatgaaatactgatacacactatgacataaaggaactttgaaaacattgtgctaagagggaaaaaaaagcca

SEQ ID NO: 17 - DNA, 410 bp, Artificial Sequence
  Description of Artificial Sequence: Synthetic polynucleotide
gtgggctcaagccatcctcccaactgagcctcctgagtagctgggactacaggtgtgagccatcacactcaactggtgtagctattttagaagacaaactggcagtttctcaaaaggctaaacatacagtcatcatataatgcaacaatttcactcctaggcatatatcccagagaaatagaaatatatgtccacacaaaaacttgtacagcaatcttcatagcagcattgttcgtaatagccaatacgtggaaacaacccaaatgtccatcaactgatgaacagataaacaaaatgcagtgtgtctctaccatggaatattattcagccacagaagaaatgaaatactgatacacactatgacataaaggaactttgaaaacattgtgctaagagggaaaaaaaagcca

SEQ ID NO: 18 - DNA, 410 bp, Artificial Sequence
  Description of Artificial Sequence: Synthetic polynucleotide
gtgggctcaagccatcctcccaactgagcctcctgagtagctgggactacaggtgtgagccatcacacccaactggtgtagctattttagaagacaaactggcagtttctcaaaaggctaaacatacagtcatcatataatgcaacaatttcactcctaggcatatatcccagagaaatagaaatatatgtccacacaaaaacttatacagcaatcttcatagcagcattattcataatagccaatacgtggaaacaacccaaatgtccatcaactgatgaacagataaacaaaatgcagtgtgtctctaccatggaatattattcagccacagaagaaatgaaatactgatacacactatgacataaaggaactttgaaaacattgtgctaagaggggaaaaaaagcca